SEQUENCE LISTING (232 sequences)

SEQ ID NO: 1 - protein, 11 aa, synthetic construct
  REGION 1..11: note = Synthetic: G3I8R9_1*
VEIIANDQGNR

SEQ ID NO: 2 - protein, 14 aa, synthetic construct
  REGION 1..14: note = Synthetic: G3I8R9_2
ITPSYVAFTPEGER

SEQ ID NO: 3 - protein, 12 aa, synthetic construct
  REGION 1..12: note = Synthetic: G3I8R9_3
TWNDPSVQQDIK

SEQ ID NO: 4 - protein, 17 aa, synthetic construct
  REGION 1..17: note = Synthetic: G3I8R9_4*
VTHAVVTVPAYFNDAQR

SEQ ID NO: 5 - protein, 17 aa, synthetic construct
  REGION 1..17: note = Synthetic: G3I8R9_5
SQIFSTASDNQPTVTIK

SEQ ID NO: 6 - protein, 10 aa, synthetic construct
  REGION 1..10: note = Synthetic: G3I0W1_1*
IGAEVYHNLK

SEQ ID NO: 7 - protein, 8 aa, synthetic construct
  REGION 1..8: note = Synthetic: G3I0W1_2*
  MOD_RES 2: note = carbamidomethylated cysteine
  MOD_RES 4: note = carbamidomethylated cysteine
SCNCLLLK

SEQ ID NO: 8 - protein, 12 aa, synthetic construct
  REGION 1..12: note = Synthetic: G3I5A4_1*
GTVTAFPGFDSR

SEQ ID NO: 9 - protein, 16 aa, synthetic construct
  REGION 1..16: note = Synthetic: G3I5A4_2
GLGTDEESILTLLTSR

SEQ ID NO: 10 - protein, 8 aa, synthetic construct
  REGION 1..8: note = Synthetic: G3I5A4_3
LYDAYELK

SEQ ID NO: 11 - protein, 9 aa, synthetic construct
  REGION 1..9: note = Synthetic: G3I5A4_4*
VLTEIIASR

SEQ ID NO: 12 - protein, 8 aa, synthetic construct
  REGION 1..8: note = Synthetic: G3I5A4_5
FITIFGTR

SEQ ID NO: 13 - protein, 8 aa, synthetic construct
  REGION 1..8: note = Synthetic: G3GTT2_1*
EAVIFITK

SEQ ID NO: 14 - protein, 9 aa, synthetic construct
  REGION 1..9: note = Synthetic: G3GTT2_2*
QDWVQTYTK

SEQ ID NO: 15 - protein, 10 aa, synthetic construct
  REGION 1..10: note = Synthetic: A4URF0_1*
IDFEDIQSLK

SEQ ID NO: 16 - protein, 18 aa, synthetic construct
  REGION 1..18: note = Synthetic: A4URF0_2
  MOD_RES 8: note = carbamidomethylated cysteine
VTPPGPHCTQTEVIATLK

SEQ ID NO: 17 - protein, 13 aa, synthetic construct
  REGION 1..13: note = Synthetic: A4URF0_3*
  MOD_RES 6: note = carbamidomethylated cysteine
DGQEVCLDPEGPR

SEQ ID NO: 18 - protein, 11 aa, synthetic construct
  REGION 1..11: note = Synthetic: A0A061IFE2_1*
FGALDFLGDPR

SEQ ID NO: 19 - protein, 16 aa, synthetic construct
  REGION 1..16: note = Synthetic: A0A061IFE2_2*
AISESGVILVPGLFTK

SEQ ID NO: 20 - protein, 17 aa, synthetic construct
  REGION 1..17: note = Synthetic: A0A061IFE2_3*
ENHAFVPTVLDGVLLPK

SEQ ID NO: 21 - protein, 9 aa, synthetic construct
  REGION 1..9: note = Synthetic: A0A061IFE2_4
APEEILAEK

SEQ ID NO: 22 - protein, 12 aa, synthetic construct
  REGION 1..12: note = Synthetic: A0A061I7X9_1*
  MOD_RES 10: note = carbamidomethylated cysteine
TTTSAVIVHCLR

SEQ ID NO: 23 - protein, 7 aa, synthetic construct
  REGION 1..7: note = Synthetic: A0A061I7X9_2*
FWANFAR

SEQ ID NO: 24 - protein, 18 aa, synthetic construct
  REGION 1..18: note = Synthetic: A0A061I7X9_3*
NGNPNGEGLPHWPEYDQK

SEQ ID NO: 25 - protein, 9 aa, synthetic construct
  REGION 1..9: note = Synthetic: G3HR95_1
ESLITLIEK

SEQ ID NO: 26 - protein, 13 aa, synthetic construct
  REGION 1..13: note = Synthetic: G3HR95_2*
NFGTDPEVTDLVR

SEQ ID NO: 27 - protein, 12 aa, synthetic construct
  REGION 1..12: note = Synthetic: G3HR95_3*
SQEGDSVSVVGR

SEQ ID NO: 28 - protein, 8 aa, synthetic construct
  REGION 1..8: note = Synthetic: G3HR95_4
LLVPGTYK

SEQ ID NO: 29 - protein, 10 aa, synthetic construct
  REGION 1..10: note = Synthetic: G3H0L9_1*
  MOD_RES 6: note = carbamidomethylated cysteine
EQWSNCPTIK

SEQ ID NO: 30 - protein, 11 aa, synthetic construct
  REGION 1..11: note = Synthetic: G3H0L9_2*
  MOD_RES 2: note = carbamidomethylated cysteine
SCEAGYSPSYK

SEQ ID NO: 31 - protein, 14 aa, synthetic construct
  REGION 1..14: note = Synthetic: G3H0L9_3*
HYGYTSYSVSNNEK

SEQ ID NO: 32 - protein, 17 aa, synthetic construct
  REGION 1..17: note = Synthetic: G3H0L9_4*
  MOD_RES 5: note = carbamidomethylated cysteine
GEDHCGIESEIVAGIPR

SEQ ID NO: 33 - protein, 8 aa, synthetic construct
  REGION 1..8: note = Synthetic: G3I4W7_1*
GPVSELLK

SEQ ID NO: 34 - protein, 11 aa, synthetic construct
  REGION 1..11: note = Synthetic: G3I4W7_2*
  MOD_RES 6: note = carbamidomethylated cysteine
LLDIACWIHHK

SEQ ID NO: 35 - protein, 9 aa, synthetic construct
  REGION 1..9: note = Synthetic: G3I4W7_3*
NIFSFFLNR

SEQ ID NO: 36 - protein, 22 aa, synthetic construct
  REGION 1..22: note = Synthetic: G3I4W7_4
  MOD_RES 3: note = carbamidomethylated cysteine
GGCEAIVDTGTSLLVGPVDEVK

SEQ ID NO: 37 - protein, 10 aa, synthetic construct
  REGION 1..10: note = Synthetic: G3I4W7_5*
VSSLPSVTLK

SEQ ID NO: 38 - protein, 10 aa, synthetic construct
  REGION 1..10: note = Synthetic: G3INC5_1*
LYGTNEEEWR

SEQ ID NO: 39 - protein, 13 aa, synthetic construct
  REGION 1..13: note = Synthetic: G3INC5_2
GLDSEESYPYEAK

SEQ ID NO: 40 - protein, 21 aa, synthetic construct
  REGION 1..21: note = Synthetic: G3INC5_3*
YKPEFAAANDTGYVDIPQLEK

SEQ ID NO: 41 - protein, 16 aa, synthetic construct
  REGION 1..16: note = Synthetic: Q9EPP7_1
TYPRPHEYLSPSDIPK

SEQ ID NO: 42 - protein, 9 aa, synthetic construct
  REGION 1..9: note = Synthetic: Q9EPP7_2
GVNYASITR

SEQ ID NO: 43 - protein, 11 aa, synthetic construct
  REGION 1..11: note = Synthetic: Q9EPP7_3
  MOD_RES 4: note = carbamidomethylated cysteine
  MOD_RES 7: note = carbamidomethylated cysteine
FNQCGTCTEFK

SEQ ID NO: 44 - protein, 10 aa, synthetic construct
  REGION 1..10: note = Synthetic: Q9EPP7_4*
VGDYGSLSGR

SEQ ID NO: 45 - protein, 10 aa, synthetic construct
  REGION 1..10: note = Synthetic: Q9EPP7_5*
NSWGEPWGER

SEQ ID NO: 46 - protein, 10 aa, synthetic construct
  REGION 1..10: note = Synthetic: G3HNJ3_1*
EIQNAVQGVK

SEQ ID NO: 47 - protein, 8 aa, synthetic construct
  REGION 1..8: note = Synthetic: G3HNJ3_2
RPHFLYPK

SEQ ID NO: 48 - protein, 15 aa, synthetic construct
  REGION 1..15: note = Synthetic: G3HNJ3_3*
LTQQYNELLHSLQTK

SEQ ID NO: 49 - protein, 16 aa, synthetic construct
  REGION 1..16: note = Synthetic: G3HNJ3_4*
VSTVTTHSNNSEEPSR

SEQ ID NO: 50 - protein, 17 aa, synthetic construct
  REGION 1..17: note = Synthetic: G3HNJ3_5
LFDSDPITVVLPEEVSK

SEQ ID NO: 51 - protein, 11 aa, synthetic construct
  REGION 1..11: note = Synthetic: G3I3Y6_1*
ILLADQGQSWK

SEQ ID NO: 52 - protein, 10 aa, synthetic construct
  REGION 1..10: note = Synthetic: G3I3Y6_2*
  MOD_RES 3: note = carbamidomethylated cysteine
STCLYGQLPK

SEQ ID NO: 53 - protein, 16 aa, synthetic construct
  REGION 1..16: note = Synthetic: G3I3Y6_3*
FEDGDLTLYQSNAILR

SEQ ID NO: 54 - protein, 8 aa, synthetic construct
  REGION 1..8: note = Synthetic: G3I3Y6_4*
YITLIYTK

SEQ ID NO: 55 - protein, 20 aa, synthetic construct
  REGION 1..20: note = Synthetic: G3I3Y6_5
ALPGHLKPFETLLSQNQGGK

SEQ ID NO: 56 - protein, 8 aa, synthetic construct
  REGION 1..8: note = Synthetic: G3H6V7_1*
DFTDIESK

SEQ ID NO: 57 - protein, 13 aa, synthetic construct
  REGION 1..13: note = Synthetic: G3H6V7_2
AQQHYPVSAGYTK

SEQ ID NO: 58 - protein, 19 aa, synthetic construct
  REGION 1..19: note = Synthetic: G3H6V7_3
ITGLDPAGPNFEYAEAPSR

SEQ ID NO: 59 - protein, 10 aa, synthetic construct
  REGION 1..10: note = Synthetic: G3H6V7_4*
GLGDVDQLVK

SEQ ID NO: 60 - protein, 17 aa, synthetic construct
  REGION 1..17: note = Synthetic: G3H6V7_5
SIHLFIDSLLNEENPSK

SEQ ID NO: 61 - protein, 7 aa, synthetic construct
  REGION 1..7: note = Synthetic: G3HQY6_1*
GNTWSLK

SEQ ID NO: 62 - protein, 13 aa, synthetic construct
  REGION 1..13: note = Synthetic: G3HQY6_2
WPEVIIEDLFGHK

SEQ ID NO: 63 - protein, 9 aa, synthetic construct
  REGION 1..9: note = Synthetic: G3HQY6_3
  MOD_RES 7: note = carbamidomethylated cysteine
WLSTHVCNR

SEQ ID NO: 64 - protein, 18 aa, synthetic construct
  REGION 1..18: note = Synthetic: G3HQY6_4*
VNVYTSHSPAGTSVQNLR

SEQ ID NO: 65 - protein, 7 aa, synthetic construct
  REGION 1..7: note = Synthetic: G3HQY6_5*
HWGQIAK

SEQ ID NO: 66 - protein, 18 aa, synthetic construct
  REGION 1..18: note = Synthetic: G3HKV9_1*
HPPVVLVPGDLGNQLEAK

SEQ ID NO: 67 - protein, 11 aa, synthetic construct
  REGION 1..11: note = Synthetic: G3HKV9_2*
ATQFPDGVDVR

SEQ ID NO: 68 - protein, 8 aa, synthetic construct
  REGION 1..8: note = Synthetic: G3HKV9_3
IPVIGPLK

SEQ ID NO: 69 - protein, 10 aa, synthetic construct
  REGION 1..10: note = Synthetic: G3HKV9_4
DYHQFFQDIR

SEQ ID NO: 70 - protein, 9 aa, synthetic construct
  REGION 1..9: note = Synthetic: G3HRK9_1*
LEDITEALR

SEQ ID NO: 71 - protein, 12 aa, synthetic construct
  REGION 1..12: note = Synthetic: G3HRK9_2*
ILNLPSTLSPSR

SEQ ID NO: 72 - protein, 17 aa, synthetic construct
  REGION 1..17: note = Synthetic: G3HRK9_3
GDYVWTVTDSGPGPLFR

SEQ ID NO: 73 - protein, 8 aa, synthetic construct
  REGION 1..8: note = Synthetic: G3HRK9_4
TGWIHFFK

SEQ ID NO: 74 - protein, 16 aa, synthetic construct
  REGION 1..16: note = Synthetic: G3HRK9_5
VEPNLDAALYWPVNQK

SEQ ID NO: 75 - protein, 9 aa, synthetic construct
  REGION 1..9: note = Synthetic: P14851_1
VSFELFADK

SEQ ID NO: 76 - protein, 9 aa, synthetic construct
  REGION 1..9: note = Synthetic: P14851_2*
FEDENFILK

SEQ ID NO: 77 - protein, 7 aa, synthetic construct
  REGION 1..7: note = Synthetic: P14851_3*
TEWLDGK

SEQ ID NO: 78 - protein, 9 aa, synthetic construct
  REGION 1..9: note = Synthetic: Q9JKY1_1
IGYPAPNFK

SEQ ID NO: 79 - protein, 8 aa, synthetic construct
  REGION 1..8: note = Synthetic: Q9JKY1_2
  MOD_RES 3: note = carbamidomethylated cysteine
DICLSEYR

SEQ ID NO: 80 - protein, 8 aa, synthetic construct
  REGION 1..8: note = Synthetic: Q9JKY1_3
ADEGISFR

SEQ ID NO: 81 - protein, 8 aa, synthetic construct
  REGION 1..8: note = Synthetic: Q9JKY1_4*
GLFIIDDK

SEQ ID NO: 82 - protein, 7 aa, synthetic construct
  REGION 1..7: note = Synthetic: Q9JKY1_5*
SVDEILR

SEQ ID NO: 83 - protein, 12 aa, synthetic construct
  REGION 1..12: note = Synthetic: G3I6T1_1*
SVLLDAASGQLR

SEQ ID NO: 84 - protein, 7 aa, synthetic construct
  REGION 1..7: note = Synthetic: G3I6T1_2*
LTLLQLK

SEQ ID NO: 85 - protein, 13 aa, synthetic construct
  REGION 1..13: note = Synthetic: G3I6T1_3*
  MOD_RES 8: note = carbamidomethylated cysteine
LSLGSGSCSAIIK

SEQ ID NO: 86 - protein, 12 aa, synthetic construct
  REGION 1..12: note = Synthetic: G3I6T1_4
AFIPNGPSPGSR

SEQ ID NO: 87 - protein, 8 aa, synthetic construct
  REGION 1..8: note = Synthetic: G3I6T1_5*
VTSFSLAK

SEQ ID NO: 88 - protein, 8 aa, synthetic construct
  REGION 1..8: note = Synthetic: G3IIE7_1
SAQFFNYK

SEQ ID NO: 89 - protein, 16 aa, synthetic construct
  REGION 1..16: note = Synthetic: G3IIE7_2*
FLGSGGFIGYAPNLNK

SEQ ID NO: 90 - protein, 18 aa, synthetic construct
  REGION 1..18: note = Synthetic: G3IIE7_3*
NLAYDTLPVVIHGNGPTK

SEQ ID NO: 91 - protein, 13 aa, synthetic construct
  REGION 1..13: note = Synthetic: G3IIE7_4
FLAEHGTEYQSVK

SEQ ID NO: 92 - protein, 7 aa, synthetic construct
  REGION 1..7: note = Synthetic: G3IIE7_5
LLIEQNK

SEQ ID NO: 93 - protein, 16 aa, synthetic construct
  REGION 1..16: note = Synthetic: Q8R4U2_1
VDATEESDLAQQYGVR

SEQ ID NO: 94 - protein, 17 aa, synthetic construct
  REGION 1..17: note = Synthetic: Q8R4U2_2*
HNQLPLVIEFTEQTAPK

SEQ ID NO: 95 - protein, 15 aa, synthetic construct
  REGION 1..15: note = Synthetic: Q8R4U2_3
ILFIFIDSDHTDNQR

SEQ ID NO: 96 - protein, 12 aa, synthetic construct
  REGION 1..12: note = Synthetic: Q8R4U2_4*
YKPESDELTAEK

SEQ ID NO: 97 - protein, 9 aa, synthetic construct
  REGION 1..9: note = Synthetic: Q8R4U2_5
TVIDYNGER

SEQ ID NO: 98 - protein, 8 aa, synthetic construct
  REGION 1..8: note = Synthetic: G3HC31_1*
LQDAEIAR

SEQ ID NO: 99 - protein, 7 aa, synthetic construct
  REGION 1..7: note = Synthetic: G3HC31_2*
LMDDLDR

SEQ ID NO: 100 - protein, 25 aa, synthetic construct
  REGION 1..25: note = Synthetic: G3HC31_3
DQEVNFQEYVAFLGALALIYNEALK

SEQ ID NO: 101 - protein, 11 aa, synthetic construct
  REGION 1..11: note = Synthetic: G3H3Q1_1*
GDLGIEIPAEK

SEQ ID NO: 102 - protein, 9 aa, synthetic construct
  REGION 1..9: note = Synthetic: G3H3Q1_2*
GDYPLEAVR

SEQ ID NO: 103 - protein, 8 aa, synthetic construct
  REGION 1..8: note = Synthetic: G3H3Q1_3
APIIAVTR

SEQ ID NO: 104 - protein, 8 aa, synthetic construct
  REGION 1..8: note = Synthetic: G3H3Q1_4
  MOD_RES 7: note = carbamidomethylated cysteine
GIFPVLCK

SEQ ID NO: 105 - protein, 14 aa, synthetic construct
  REGION 1..14: note = Synthetic: G3H3Q1_5
DAVQDAWAEDVDLR

SEQ ID NO: 106 - protein, 8 aa, synthetic construct
  REGION 1..8: note = Synthetic: G3IBF4_1*
LPVLLLGR

SEQ ID NO: 107 - protein, 13 aa, synthetic construct
  REGION 1..13: note = Synthetic: G3IBF4_2
VTAGISFAIPSDK

SEQ ID NO: 108 - protein, 8 aa, synthetic construct
  REGION 1..8: note = Synthetic: G3IBF4_3*
FLTESHDR

SEQ ID NO: 109 - protein, 26 aa, synthetic construct
  REGION 1..26: note = Synthetic: G3IBF4_4
DFPDVLSGAYIIEVIPDTPAEAGGLK

SEQ ID NO: 110 - protein, 12 aa, synthetic construct
  REGION 1..12: note = Synthetic: A0A3L7HS03_1*
ELAVAAAYQSVR

SEQ ID NO: 111 - protein, 14 aa, synthetic construct
  REGION 1..14: note = Synthetic: A0A3L7HS03_2*
WHQTADFGFVPNLK

SEQ ID NO: 112 - protein, 9 aa, synthetic construct
  REGION 1..9: note = Synthetic: A0A3L7HS03_3*
LLSLTYDQK

SEQ ID NO: 113 - protein, 10 aa, synthetic construct
  REGION 1..10: note = Synthetic: A0A3L7HS03_4*
  MOD_RES 6..7: note = carbamidomethylated cysteine
SFEISCCSDR

SEQ ID NO: 114 - protein, 11 aa, synthetic construct
  REGION 1..11: note = Synthetic: A0A3L7HS03_5
  MOD_RES 8: note = carbamidomethylated cysteine
YAWTTWPCDYK

SEQ ID NO: 115 - protein, 14 aa, synthetic construct
  REGION 1..14: note = Synthetic: G3IA12_1*
DNVLHVEINGISPK

SEQ ID NO: 116 - protein, 10 aa, synthetic construct
  REGION 1..10: note = Synthetic: G3IA12_2*
  MOD_RES 5: note = carbamidomethylated cysteine
  MOD_RES 7: note = carbamidomethylated cysteine
FSAHCSCDSK

SEQ ID NO: 117 - protein, 11 aa, synthetic construct
  REGION 1..11: note = Synthetic: G3I8R9_1*
  MOD_RES 11: note = heavy Arginine
VEIIANDQGNR

SEQ ID NO: 118 - protein, 14 aa, synthetic construct
  REGION 1..14: note = Synthetic: G3I8R9_2
  MOD_RES 14: note = heavy Arginine
ITPSYVAFTPEGER

SEQ ID NO: 119 - protein, 12 aa, synthetic construct
  REGION 1..12: note = Synthetic: G3I8R9_3
  MOD_RES 12: note = heavy L-Lysine
TWNDPSVQQDIK

SEQ ID NO: 120 - protein, 17 aa, synthetic construct
  REGION 1..17: note = Synthetic: G3I8R9_4*
  MOD_RES 17: note = heavy Arginine
VTHAVVTVPAYFNDAQR

SEQ ID NO: 121 - protein, 17 aa, synthetic construct
  REGION 1..17: note = Synthetic: G3I8R9_5
  MOD_RES 17: note = heavy L-Lysine
SQIFSTASDNQPTVTIK

SEQ ID NO: 122 - protein, 10 aa, synthetic construct
  REGION 1..10: note = Synthetic: G3I0W1_1*
  MOD_RES 10: note = heavy L-Lysine
IGAEVYHNLK

SEQ ID NO: 123 - protein, 8 aa, synthetic construct
  REGION 1..8: note = Synthetic: G3I0W1_2*
  MOD_RES 2: note = carbamidomethylated cysteine
  MOD_RES 4: note = carbamidomethylated cysteine
  MOD_RES 8: note = heavy L-Lysine
SCNCLLLK

SEQ ID NO: 124 - protein, 12 aa, synthetic construct
  REGION 1..12: note = Synthetic: G3I5A4_1*
  MOD_RES 12: note = heavy Arginine
GTVTAFPGFDSR

SEQ ID NO: 125 - protein, 16 aa, synthetic construct
  REGION 1..16: note = Synthetic: G3I5A4_2
  MOD_RES 16: note = heavy Arginine
GLGTDEESILTLLTSR

SEQ ID NO: 126 - protein, 8 aa, synthetic construct
  REGION 1..8: note = Synthetic: G3I5A4_3
  MOD_RES 8: note = heavy L-Lysine
LYDAYELK

SEQ ID NO: 127 - protein, 9 aa, synthetic construct
  REGION 1..9: note = Synthetic: G3I5A4_4*
  MOD_RES 9: note = heavy Arginine
VLTEIIASR

SEQ ID NO: 128 - protein, 8 aa, synthetic construct
  REGION 1..8: note = Synthetic: G3I5A4_5
  MOD_RES 8: note = heavy Arginine
FITIFGTR

SEQ ID NO: 129 - protein, 8 aa, synthetic construct
  REGION 1..8: note = Synthetic: G3GTT2_1*
  MOD_RES 8: note = heavy L-Lysine
EAVIFITK

SEQ ID NO: 130 - protein, 9 aa, synthetic construct
  REGION 1..9: note = Synthetic: G3GTT2_2*
  MOD_RES 9: note = heavy L-Lysine
QDWVQTYTK

SEQ ID NO: 131 - protein, 10 aa, synthetic construct
  REGION 1..10: note = Synthetic: A4URF0_1*
  MOD_RES 10: note = heavy L-Lysine
IDFEDIQSLK

SEQ ID NO: 132 - protein, 18 aa, synthetic construct
  REGION 1..18: note = Synthetic: A4URF0_2
  MOD_RES 8: note = carbamidomethylated cysteine
  MOD_RES 18: note = heavy L-Lysine
VTPPGPHCTQTEVIATLK

SEQ ID NO: 133 - protein, 13 aa, synthetic construct
  REGION 1..13: note = Synthetic: A4URF0_3*
  MOD_RES 6: note = carbamidomethylated cysteine
  MOD_RES 13: note = heavy Arginine
DGQEVCLDPEGPR

SEQ ID NO: 134 - protein, 11 aa, synthetic construct
  REGION 1..11: note = Synthetic: A0A061IFE2_1*
  MOD_RES 11: note = heavy Arginine
FGALDFLGDPR

SEQ ID NO: 135 - protein, 16 aa, synthetic construct
  REGION 1..16: note = Synthetic: A0A061IFE2_2*
  MOD_RES 16: note = heavy L-Lysine
AISESGVILVPGLFTK

SEQ ID NO: 136 - protein, 17 aa, synthetic construct
  REGION 1..17: note = Synthetic: A0A061IFE2_3*
  MOD_RES 17: note = heavy L-Lysine
ENHAFVPTVLDGVLLPK

SEQ ID NO: 137 - protein, 9 aa, synthetic construct
  REGION 1..9: note = Synthetic: A0A061IFE2_4
  MOD_RES 9: note = heavy L-Lysine
APEEILAEK

SEQ ID NO: 138 - protein, 12 aa, synthetic construct
  REGION 1..12: note = Synthetic: A0A061I7X9_1*
  MOD_RES 10: note = carbamidomethylated cysteine
  MOD_RES 12: note = heavy Arginine
TTTSAVIVHCLR

SEQ ID NO: 139 - protein, 7 aa, synthetic construct
  REGION 1..7: note = Synthetic: A0A061I7X9_2*
  MOD_RES 7: note = heavy Arginine
FWANFAR

SEQ ID NO: 140 - protein, 18 aa, synthetic construct
  REGION 1..18: note = Synthetic: A0A061I7X9_3*
  MOD_RES 18: note = heavy L-Lysine
NGNPNGEGLPHWPEYDQK

SEQ ID NO: 141 - protein, 9 aa, synthetic construct
  REGION 1..9: note = Synthetic: G3HR95_1
  MOD_RES 9: note = heavy L-Lysine
ESLITLIEK

SEQ ID NO: 142 - protein, 13 aa, synthetic construct
  REGION 1..13: note = Synthetic: G3HR95_2*
  MOD_RES 13: note = heavy Arginine
NFGTDPEVTDLVR

SEQ ID NO: 143 - protein, 12 aa, synthetic construct
  REGION 1..12: note = Synthetic: G3HR95_3*
  MOD_RES 12: note = heavy Arginine
SQEGDSVSVVGR

SEQ ID NO: 144 - protein, 8 aa, synthetic construct
  REGION 1..8: note = Synthetic: G3HR95_4
  MOD_RES 8: note = heavy L-Lysine
LLVPGTYK

SEQ ID NO: 145 - protein, 10 aa, synthetic construct
  REGION 1..10: note = Synthetic: G3H0L9_1*
  MOD_RES 6: note = carbamidomethylated cysteine
  MOD_RES 10: note = heavy L-Lysine
EQWSNCPTIK

SEQ ID NO: 146 - protein, 11 aa, synthetic construct
  REGION 1..11: note = Synthetic: G3H0L9_2*
  MOD_RES 2: note = carbamidomethylated cysteine
  MOD_RES 11: note = heavy L-Lysine
SCEAGYSPSYK

SEQ ID NO: 147 - protein, 14 aa, synthetic construct
  REGION 1..14: note = Synthetic: G3H0L9_3*
  MOD_RES 14: note = heavy L-Lysine
HYGYTSYSVSNNEK

SEQ ID NO: 148 - protein, 17 aa, synthetic construct
  REGION 1..17: note = Synthetic: G3H0L9_4*
  MOD_RES 5: note = carbamidomethylated cysteine
  MOD_RES 17: note = heavy Arginine
GEDHCGIESEIVAGIPR

SEQ ID NO: 149 - protein, 8 aa, synthetic construct
  REGION 1..8: note = Synthetic: G3I4W7_1*
  MOD_RES 8: note = heavy L-Lysine
GPVSELLK

SEQ ID NO: 150 - protein, 11 aa, synthetic construct
  REGION 1..11: note = Synthetic: G3I4W7_2*
  MOD_RES 6: note = carbamidomethylated cysteine
  MOD_RES 11: note = heavy L-Lysine
LLDIACWIHHK

SEQ ID NO: 151 - protein, 9 aa, synthetic construct
  REGION 1..9: note = Synthetic: G3I4W7_3*
  MOD_RES 9: note = heavy Arginine
NIFSFFLNR

SEQ ID NO: 152 - protein, 22 aa, synthetic construct
  REGION 1..22: note = Synthetic: G3I4W7_4
  MOD_RES 3: note = carbamidomethylated cysteine
  MOD_RES 22: note = heavy L-Lysine
GGCEAIVDTGTSLLVGPVDEVK

SEQ ID NO: 153 - protein, 10 aa, synthetic construct
  REGION 1..10: note = Synthetic: G3I4W7_5*
  MOD_RES 10: note = heavy L-Lysine
VSSLPSVTLK

SEQ ID NO: 154 - protein, 10 aa, synthetic construct
  REGION 1..10: note = Synthetic: G3INC5_1*
  MOD_RES 10: note = heavy Arginine
LYGTNEEEWR

SEQ ID NO: 155 - protein, 13 aa, synthetic construct
  REGION 1..13: note = Synthetic: G3INC5_2
  MOD_RES 13: note = heavy L-Lysine
GLDSEESYPYEAK

SEQ ID NO: 156 - protein, 21 aa, synthetic construct
  REGION 1..21: note = Synthetic: G3INC5_3*
  MOD_RES 21: note = heavy L-Lysine
YKPEFAAANDTGYVDIPQLEK

SEQ ID NO: 157 - protein, 16 aa, synthetic construct
  REGION 1..16: note = Synthetic: Q9EPP7_1
  MOD_RES 16: note = heavy L-Lysine
TYPRPHEYLSPSDIPK

SEQ ID NO: 158 - protein, 9 aa, synthetic construct
  REGION 1..9: note = Synthetic: Q9EPP7_2
  MOD_RES 9: note = heavy Arginine
GVNYASITR

SEQ ID NO: 159 - protein, 11 aa, synthetic construct
  REGION 1..11: note = Synthetic: Q9EPP7_3
  MOD_RES 4: note = carbamidomethylated cysteine
  MOD_RES 7: note = carbamidomethylated cysteine
  MOD_RES 11: note = heavy L-Lysine
FNQCGTCTEFK

SEQ ID NO: 160 - protein, 10 aa, synthetic construct
  REGION 1..10: note = Synthetic: Q9EPP7_4*
  MOD_RES 10: note = heavy Arginine
VGDYGSLSGR

SEQ ID NO: 161 - protein, 10 aa, synthetic construct
  REGION 1..10: note = Synthetic: Q9EPP7_5*
  MOD_RES 10: note = heavy Arginine
NSWGEPWGER

SEQ ID NO: 162 - protein, 10 aa, synthetic construct
  REGION 1..10: note = Synthetic: G3HNJ3_1*
  MOD_RES 10: note = heavy L-Lysine
EIQNAVQGVK

SEQ ID NO: 163 - protein, 8 aa, synthetic construct
  REGION 1..8: note = Synthetic: G3HNJ3_2
  MOD_RES 8: note = heavy L-Lysine
RPHFLYPK

SEQ ID NO: 164 - protein, 15 aa, synthetic construct
  REGION 1..15: note = Synthetic: G3HNJ3_3*
  MOD_RES 15: note = heavy L-Lysine
LTQQYNELLHSLQTK

SEQ ID NO: 165 - protein, 16 aa, synthetic construct
  REGION 1..16: note = Synthetic: G3HNJ3_4*
  MOD_RES 16: note = heavy Arginine
VSTVTTHSNNSEEPSR

SEQ ID NO: 166 - protein, 17 aa, synthetic construct
  REGION 1..17: note = Synthetic: G3HNJ3_5
  MOD_RES 17: note = heavy L-Lysine
LFDSDPITVVLPEEVSK

SEQ ID NO: 167 - protein, 11 aa, synthetic construct
  REGION 1..11: note = Synthetic: G3I3Y6_1*
  MOD_RES 11: note = heavy L-Lysine
ILLADQGQSWK

SEQ ID NO: 168 - protein, 10 aa, synthetic construct
  REGION 1..10: note = Synthetic: G3I3Y6_2*
  MOD_RES 3: note = carbamidomethylated cysteine
  MOD_RES 10: note = heavy L-Lysine
STCLYGQLPK

SEQ ID NO: 169 - protein, 16 aa, synthetic construct
  REGION 1..16: note = Synthetic: G3I3Y6_3*
  MOD_RES 16: note = heavy Arginine
FEDGDLTLYQSNAILR

SEQ ID NO: 170 - protein, 8 aa, synthetic construct
  REGION 1..8: note = Synthetic: G3I3Y6_4*
  MOD_RES 8: note = heavy L-Lysine
YITLIYTK

SEQ ID NO: 171 - protein, 20 aa, synthetic construct
  REGION 1..20: note = Synthetic: G3I3Y6_5
  MOD_RES 20: note = heavy L-Lysine
ALPGHLKPFETLLSQNQGGK

SEQ ID NO: 172 - protein, 8 aa, synthetic construct
  REGION 1..8: note = Synthetic: G3H6V7_1*
  MOD_RES 8: note = heavy L-Lysine
DFTDIESK

SEQ ID NO: 173 - protein, 13 aa, synthetic construct
  REGION 1..13: note = Synthetic: G3H6V7_2
  MOD_RES 13: note = heavy L-Lysine
AQQHYPVSAGYTK

SEQ ID NO: 174 - protein, 19 aa, synthetic construct
  REGION 1..19: note = Synthetic: G3H6V7_3
  MOD_RES 19: note = heavy Arginine
ITGLDPAGPNFEYAEAPSR

SEQ ID NO: 175 - protein, 10 aa, synthetic construct
  REGION 1..10: note = Synthetic: G3H6V7_4*
  MOD_RES 10: note = heavy L-Lysine
GLGDVDQLVK

SEQ ID NO: 176 - protein, 17 aa, synthetic construct
  REGION 1..17: note = Synthetic: G3H6V7_5
  MOD_RES 17: note = heavy L-Lysine
SIHLFIDSLLNEENPSK

SEQ ID NO: 177 - protein, 7 aa, synthetic construct
  REGION 1..7: note = Synthetic: G3HQY6_1*
  MOD_RES 7: note = heavy L-Lysine
GNTWSLK

SEQ ID NO: 178 - protein, 13 aa, synthetic construct
  REGION 1..13: note = Synthetic: G3HQY6_2
  MOD_RES 13: note = heavy L-Lysine
WPEVIIEDLFGHK

SEQ ID NO: 179 - protein, 9 aa, synthetic construct
  REGION 1..9: note = Synthetic: G3HQY6_3
  MOD_RES 7: note = carbamidomethylated cysteine
  MOD_RES 9: note = heavy Arginine
WLSTHVCNR

SEQ ID NO: 180 - protein, 18 aa, synthetic construct
  REGION 1..18: note = Synthetic: G3HQY6_4*
  MOD_RES 18: note = heavy Arginine
VNVYTSHSPAGTSVQNLR

SEQ ID NO: 181 - protein, 7 aa, synthetic construct
  REGION 1..7: note = Synthetic: G3HQY6_5*
  MOD_RES 7: note = heavy L-Lysine
HWGQIAK

SEQ ID NO: 182 - protein, 18 aa, synthetic construct
  REGION 1..18: note = Synthetic: G3HKV9_1*
  MOD_RES 18: note = heavy L-Lysine
HPPVVLVPGDLGNQLEAK

SEQ ID NO: 183 - protein, 11 aa, synthetic construct
  REGION 1..11: note = Synthetic: G3HKV9_2*
  MOD_RES 11: note = heavy Arginine
ATQFPDGVDVR

SEQ ID NO: 184 - protein, 8 aa, synthetic construct
  REGION 1..8: note = Synthetic: G3HKV9_3
  MOD_RES 8: note = heavy L-Lysine
IPVIGPLK

SEQ ID NO: 185 - protein, 10 aa, synthetic construct
  REGION 1..10: note = Synthetic: G3HKV9_4
  MOD_RES 10: note = heavy Arginine
DYHQFFQDIR

SEQ ID NO: 186 - protein, 9 aa, synthetic construct
  REGION 1..9: note = Synthetic: G3HRK9_1*
  MOD_RES 9: note = heavy Arginine
LEDITEALR

SEQ ID NO: 187 - protein, 12 aa, synthetic construct
  REGION 1..12: note = Synthetic: G3HRK9_2*
  MOD_RES 12: note = heavy Arginine
ILNLPSTLSPSR

SEQ ID NO: 188 - protein, 17 aa, synthetic construct
  REGION 1..17: note = Synthetic: G3HRK9_3
  MOD_RES 17: note = heavy Arginine
GDYVWTVTDSGPGPLFR

SEQ ID NO: 189 - protein, 8 aa, synthetic construct
  REGION 1..8: note = Synthetic: G3HRK9_4
  MOD_RES 8: note = heavy L-Lysine
TGWIHFFK

SEQ ID NO: 190 - protein, 16 aa, synthetic construct
  REGION 1..16: note = Synthetic: G3HRK9_5
  MOD_RES 16: note = heavy L-Lysine
VEPNLDAALYWPVNQK

SEQ ID NO: 191 - protein, 9 aa, synthetic construct
  REGION 1..9: note = Synthetic: P14851_1
  MOD_RES 9: note = heavy L-Lysine
VSFELFADK

SEQ ID NO: 192 - protein, 9 aa, synthetic construct
  REGION 1..9: note = Synthetic: P14851_2*
  MOD_RES 9: note = heavy L-Lysine
FEDENFILK

SEQ ID NO: 193 - protein, 7 aa, synthetic construct
  REGION 1..7: note = Synthetic: P14851_3*
  MOD_RES 7: note = heavy L-Lysine
TEWLDGK

SEQ ID NO: 194 - protein, 9 aa, synthetic construct
  REGION 1..9: note = Synthetic: Q9JKY1_1
  MOD_RES 9: note = heavy L-Lysine
IGYPAPNFK

SEQ ID NO: 195 - protein, 8 aa, synthetic construct
  REGION 1..8: note = Synthetic: Q9JKY1_2
  MOD_RES 3: note = carbamidomethylated cysteine
  MOD_RES 8: note = heavy Arginine
DICLSEYR

SEQ ID NO: 196 - protein, 8 aa, synthetic construct
  REGION 1..8: note = Synthetic: Q9JKY1_3
  MOD_RES 8: note = heavy Arginine
ADEGISFR

SEQ ID NO: 197 - protein, 8 aa, synthetic construct
  REGION 1..8: note = Synthetic: Q9JKY1_4*
  MOD_RES 8: note = heavy L-Lysine
GLFIIDDK

SEQ ID NO: 198 - protein, 7 aa, synthetic construct
  REGION 1..7: note = Synthetic: Q9JKY1_5*
  MOD_RES 7: note = heavy Arginine
SVDEILR

SEQ ID NO: 199 - protein, 12 aa, synthetic construct
  REGION 1..12: note = Synthetic: G3I6T1_1*
  MOD_RES 12: note = heavy Arginine
SVLLDAASGQLR

SEQ ID NO: 200 - protein, 7 aa, synthetic construct
  REGION 1..7: note = Synthetic: G3I6T1_2*
  MOD_RES 7: note = heavy L-Lysine
LTLLQLK

SEQ ID NO: 201 - protein, 13 aa, synthetic construct
  REGION 1..13: note = Synthetic: G3I6T1_3*
  MOD_RES 8: note = carbamidomethylated cysteine
  MOD_RES 13: note = heavy L-Lysine
LSLGSGSCSAIIK

SEQ ID NO: 202 - protein, 12 aa, synthetic construct
  REGION 1..12: note = Synthetic: G3I6T1_4
  MOD_RES 12: note = heavy Arginine
AFIPNGPSPGSR

SEQ ID NO: 203 - protein, 8 aa, synthetic construct
  REGION 1..8: note = Synthetic: G3I6T1_5*
  MOD_RES 8: note = heavy L-Lysine
VTSFSLAK

SEQ ID NO: 204 - protein, 8 aa, synthetic construct
  REGION 1..8: note = Synthetic: G3IIE7_1
  MOD_RES 8: note = heavy L-Lysine
SAQFFNYK

SEQ ID NO: 205 - protein, 16 aa, synthetic construct
  REGION 1..16: note = Synthetic: G3IIE7_2*
  MOD_RES 16: note = heavy L-Lysine
FLGSGGFIGYAPNLNK

SEQ ID NO: 206 - protein, 18 aa, synthetic construct
  REGION 1..18: note = Synthetic: G3IIE7_3*
  MOD_RES 18: note = heavy L-Lysine
NLAYDTLPVVIHGNGPTK

SEQ ID NO: 207 - protein, 13 aa, synthetic construct
  REGION 1..13: note = Synthetic: G3IIE7_4
  MOD_RES 13: note = heavy L-Lysine
FLAEHGTEYQSVK

SEQ ID NO: 208 - protein, 7 aa, synthetic construct
  REGION 1..7: note = Synthetic: G3IIE7_5
  MOD_RES 7: note = heavy L-Lysine
LLIEQNK

SEQ ID NO: 209 - protein, 16 aa, synthetic construct
  REGION 1..16: note = Synthetic: Q8R4U2_1
  MOD_RES 16: note = heavy Arginine
VDATEESDLAQQYGVR

SEQ ID NO: 210 - protein, 17 aa, synthetic construct
  REGION 1..17: note = Synthetic: Q8R4U2_2*
  MOD_RES 17: note = heavy L-Lysine
HNQLPLVIEFTEQTAPK

SEQ ID NO: 211 - protein, 15 aa, synthetic construct
  REGION 1..15: note = Synthetic: Q8R4U2_3
  MOD_RES 15: note = heavy Arginine
ILFIFIDSDHTDNQR

SEQ ID NO: 212 - protein, 12 aa, synthetic construct
  REGION 1..12: note = Synthetic: Q8R4U2_4*
  MOD_RES 12: note = heavy L-Lysine
YKPESDELTAEK

SEQ ID NO: 213 - protein, 9 aa, synthetic construct
  REGION 1..9: note = Synthetic: Q8R4U2_5
  MOD_RES 9: note = heavy Arginine
TVIDYNGER

SEQ ID NO: 214 - protein, 8 aa, synthetic construct
  REGION 1..8: note = Synthetic: G3HC31_1*
  MOD_RES 8: note = heavy Arginine
LQDAEIAR

SEQ ID NO: 215 - protein, 7 aa, synthetic construct
  REGION 1..7: note = Synthetic: G3HC31_2*
  MOD_RES 7: note = heavy Arginine
LMDDLDR

SEQ ID NO: 216 - protein, 25 aa, synthetic construct
  REGION 1..25: note = Synthetic: G3HC31_3
  MOD_RES 25: note = heavy L-Lysine
DQEVNFQEYVAFLGALALIYNEALK

SEQ ID NO: 217 - protein, 11 aa, synthetic construct
  REGION 1..11: note = Synthetic: G3H3Q1_1*
  MOD_RES 11: note = heavy L-Lysine
GDLGIEIPAEK

SEQ ID NO: 218 - protein, 9 aa, synthetic construct
  REGION 1..9: note = Synthetic: G3H3Q1_2*
  MOD_RES 9: note = heavy Arginine
GDYPLEAVR

SEQ ID NO: 219 - protein, 8 aa, synthetic construct
  REGION 1..8: note = Synthetic: G3H3Q1_3
  MOD_RES 8: note = heavy Arginine
APIIAVTR

SEQ ID NO: 220 - protein, 8 aa, synthetic construct
  REGION 1..8: note = Synthetic: G3H3Q1_4
  MOD_RES 7: note = carbamidomethylated cysteine
  MOD_RES 8: note = heavy L-Lysine
GIFPVLCK

SEQ ID NO: 221 - protein, 14 aa, synthetic construct
  REGION 1..14: note = Synthetic: G3H3Q1_5
  MOD_RES 14: note = heavy Arginine
DAVQDAWAEDVDLR

SEQ ID NO: 222 - protein, 8 aa, synthetic construct
  REGION 1..8: note = Synthetic: G3IBF4_1*
  MOD_RES 8: note = heavy Arginine
LPVLLLGR

SEQ ID NO: 223 - protein, 13 aa, synthetic construct
  REGION 1..13: note = Synthetic: G3IBF4_2
  MOD_RES 13: note = heavy L-Lysine
VTAGISFAIPSDK

SEQ ID NO: 224 - protein, 8 aa, synthetic construct
  REGION 1..8: note = Synthetic: G3IBF4_3*
  MOD_RES 8: note = heavy Arginine
FLTESHDR

SEQ ID NO: 225 - protein, 26 aa, synthetic construct
  REGION 1..26: note = Synthetic: G3IBF4_4
  MOD_RES 26: note = heavy L-Lysine
DFPDVLSGAYIIEVIPDTPAEAGGLK

SEQ ID NO: 226 - protein, 12 aa, synthetic construct
  REGION 1..12: note = Synthetic: A0A3L7HS03_1*
  MOD_RES 12: note = heavy Arginine
ELAVAAAYQSVR

SEQ ID NO: 227 - protein, 14 aa, synthetic construct
  REGION 1..14: note = Synthetic: A0A3L7HS03_2*
  MOD_RES 14: note = heavy L-Lysine
WHQTADFGFVPNLK

SEQ ID NO: 228 - protein, 9 aa, synthetic construct
  REGION 1..9: note = Synthetic: A0A3L7HS03_3*
  MOD_RES 9: note = heavy L-Lysine
LLSLTYDQK

SEQ ID NO: 229 - protein, 10 aa, synthetic construct
  REGION 1..10: note = Synthetic: A0A3L7HS03_4*
  MOD_RES 6..7: note = carbamidomethylated cysteine
  MOD_RES 10: note = heavy Arginine
SFEISCCSDR

SEQ ID NO: 230 - protein, 11 aa, synthetic construct
  REGION 1..11: note = Synthetic: A0A3L7HS03_5
  MOD_RES 8: note = carbamidomethylated cysteine
  MOD_RES 11: note = heavy L-Lysine
YAWTTWPCDYK

SEQ ID NO: 231 - protein, 14 aa, synthetic construct
  REGION 1..14: note = Synthetic: G3IA12_1*
  MOD_RES 14: note = heavy L-Lysine
DNVLHVEINGISPK

SEQ ID NO: 232 - protein, 10 aa, synthetic construct
  REGION 1..10: note = Synthetic: G3IA12_2*
  MOD_RES 5: note = carbamidomethylated cysteine
  MOD_RES 7: note = carbamidomethylated cysteine
  MOD_RES 10: note = heavy L-Lysine
FSAHCSCDSK

(19) United States
(12) Patent Application Publication
(10) Pub. No.: US 2026/0309719 A1
(43) Pub. Date: Oct. 8, 2026

(54) METHODS AND COMPOSITIONS FOR ANALYZING HOST CELL PROTEINS

(71) Applicant: LIFE TECHNOLOGIES CORPORATION, Carlsbad, CA (US)

(72) Inventors: Jae H. CHOI, Rockton, IL (US); Kay OPPERMAN, Beaufort, SC (US); Bhavin Bharat PATEL, Loves Park, IL (US)

(21) Appl. No.: 19/481,445

(22) PCT Filed: May 24, 2024
(86) PCT No.: PCT/US2024/031067

§ 371 Date: Nov. 4, 2025

Related U.S. Application Data
(60) Provisional application No. 63/469,258, filed on May 26, 2023.
(60) Provisional application No. 63/563,607, filed on Mar. 11, 2024.

(51) Int. Cl. G01N 33/68; G01N 33/543

(52) U.S. Cl. CPC G01N 33/6848; G01N 33/54313

(57) ABSTRACT

Improved sample preparation methods for detecting and/or quantifying host cell proteins in a sample are provided. The methods include contacting a sample with a solid support including an immunoglobulin binding protein at a pH of about 8-10 to produce a sample including the antibody or antibody fragment bound to the solid support. The methods also include contacting the sample including the antibody or antibody fragment bound to the solid support with a protease agent to produce partially digested antibody and target proteins and separating the sample into a first fraction including the antibody or antibody fragment bound to the solid support, and a second fraction including a supernatant including the partially digested target proteins. The methods also include processing the supernatant and detecting the one or more target proteins, for example using mass spectrometry. Compositions and kits for detecting host cell proteins are also provided.

### RELATED APPLICATIONS

This application claims priority to and the benefit under each of U.S. Provisional Application No. 63/469,258, filed May 26, 2023, and U.S. Provisional Application No. 63/563,607, filed Mar. 11, 2024. The entire contents of each of the aforementioned applications are herein incorporated by reference in their entirety.

### SEQUENCE LISTING

The application contains a Sequence Listing which has been submitted electronically in. XML format and is hereby incorporated by reference in its entirety. Said .XML copy, created on May 24, 2024, is named “TP386565WO1.xml” and is 349,709 bytes in size. The sequence listing contained in this .XML file is part of the specification and is hereby incorporated by reference herein in its entirety.

### FIELD

This disclosure relates to methods of preparing samples and compositions for analysis of host cell proteins in a biological sample, particularly for analysis using mass spectrometry.

### BACKGROUND

Host cell proteins (HCPs) are a heterogeneous mixture of impurities found in recombinant biotherapeutics. During process development of biopharmaceuticals, monitoring and controlling HCP contaminants is crucial for ensuring product safety, stability, and efficacy. HCP contaminants levels are highly variable due to differences in cell strains, cell culture media, cell growth conditions, and protein purification procedures. ELISA is the current gold standard method used to assess total HCP contamination; however, this method is unable to adequately measure the broad range of individual HCP proteins that impact product purity, toxicity and stability.

Mass spectrometry (MS) provides a potential alternative method to detect and quantify HCPs. However, challenges in HCP detection by MS include the large dynamic range created by large amounts of drug substance in the sample (such as about 10-100 mg antibody), but extremely low amounts of HCPs, making it difficult to detect the HCPs, and a lack of validated heavy peptide standard for MS-based targeted HCP assays. In addition, removal of antibodies with immuno-capture also captures HCPs, preventing selective antibody removal from the sample. Thus, there is a need for improved methods for detection and quantification of HCPs.

### SUMMARY

Provided herein are improved sample preparation methods that selectively reduce the amount of antibody in a sample compared to HCPs, thereby reducing dynamic range and increasing the number of HCPs detected.

In some aspects, the methods include contacting a sample (such as a sample including an antibody and one or more target proteins) with a solid support including an immunoglobulin binding protein at a pH of about 8-10, to produce a sample including the antibody or antibody fragment bound to the solid support. The methods also include contacting the sample including the antibody or antibody fragment bound to the solid support with a protease agent to produce partially digested antibody and target proteins and separating the sample including the antibody or antibody fragment bound to the solid support and the one or more target proteins into fractions including a first fraction including the antibody or antibody fragment bound to the solid support and a second fraction including a supernatant including the partially digested target proteins. The methods can include combining the first fraction and the second fraction prior to processing the supernatant. The methods also include processing the supernatant including the one or more partially digested target proteins and detecting the one or more target proteins.

In some aspects, the solid support includes a bead, for example a magnetic bead. In further aspects, the immunoglobulin binding protein includes Protein A, Protein G, Protein A/G, or Protein L. In some examples, the immunoglobulin binding protein is an alkali stable immunoglobulin binding protein. In additional examples, the solid support including the immunoglobulin binding protein is capable of high capacity immunoglobulin binding, for example, is capable of binding at least 40 mg immunoglobulin/mL.

In additional aspects, processing the supernatant including the one or more target proteins includes reducing and alkylating the one or more target proteins and digesting the reduced and alkylated target proteins, for example, with one or more protease agents to produce digested peptides. In some examples, the one or more protease agents is trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin. In one example, the protease agent is trypsin. In some aspects, the methods also include purifying the digested peptides.

In some aspects, detecting the one or more target proteins includes assaying the digested peptides via mass spectrometry to determine the presence of at least one peptide from the one or more target proteins. In one example, the mass spectrometry is liquid chromatography-mass spectrometry (LC-MS). In some aspects, the methods include adding one or more internal standard peptides of known amount to the digested peptides prior to mass spectrometry, wherein the one or more internal standard peptides have the same amino acid sequence as a target peptide, and are detectably labeled, and determining the quantity of the target peptide by comparison to the internal standard. In some examples, the detectable label is a mass-altering label. In additional examples, the quantity of one or more target proteins is determined by comparing an amount of a target peptide in the sample to the amount of the same target peptide in a control sample. In some examples, the target peptide includes or consists of the amino acid sequence of any one of SEQ ID NOs: 1-116. In other examples, the detectably labeled internal standard peptide includes or consists of the amino acid sequence of any one of SEQ ID NOs: 117-232.

In some aspects of the disclosed methods, the sample including one or more antibodies or antibody fragments is a sample prepared from a host cell culture. In some examples, the one or more target proteins include one or more host cell proteins, for example, one or more critical host cell proteins. In some examples, the one or more host cell proteins include at least 20 critical host cell proteins.

Also provided are compositions and kits that can be used for detecting one or more host cell proteins in a sample. In some aspects, provided are kits including one or more detectably labeled peptides including or consisting of the amino acid sequence of any one of SEQ ID NOs: 1-116. In some examples, the one or more detectably labeled peptides include or consist of one or more of SEQ ID NOs: 117-232. In other aspects, the kits also include a protease agent, for example one or more of trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin.

The foregoing and other features of this disclosure will become more apparent from the following detailed description of several aspects which proceeds with reference to the accompanying figures.

### BRIEF DESCRIPTION OF THE DRAWINGS

FIG. 1 is a schematic diagram illustrating exemplary procedures for sample preparation workflows. The modified sample preparation workflow described herein is illustrated on the left and the “regular” sample preparation method (using EasyPep Maxi kit) is illustrated on the right.

FIG. 2 shows the number of identified HCPs using the modified sample preparation method (blue) and regular sample preparation method (orange).

FIG. 3 shows the number of identified “critical” HCPs identified using the modified sample preparation method (gray bars) and regular sample preparation method (blue bars).

FIG. 4 shows quantification (fmol) of critical HCP peptides using the modified sample preparation method (blue) and regular sample preparation method (gray).

FIG. 5 is a graph showing critical HCP heavy peptide mixture analysis in six protein digest matrix, analyzed by PRM.

FIGS. 6A-6B show an example of extracted ion chromatograms of fragment ions of each AQUA heavy peptide (FIG. 6A) and six point standard curve (FIG. 6B) spanning 0.03 to 200 femtomole on column.

FIG. 7 shows lower limit of quantification (LLOQ) of 70 critical HCP AQUA heavy peptides.

FIG. 8 is a schematic diagram comparing the workflows for a modified EasyPep sample preparation method and a native digestion method.

FIG. 9 is a Venn diagram that illustrates the number of HCPs identified by MS analysis for samples prepared using the native digestion and modified EasyPep workflow.

FIG. 10 is a bar graph depicting the abundance levels of 28 critical HCPs measured from samples prepared using the native digestion or modified EasyPep workflow.

FIG. 11 is a schematic diagram comparing the workflows for the modified EasyPep sample preparation method and the combined Protein A depletion and native digestion preparation method.

FIG. 12 is a Venn diagram that illustrates the number of HCPs identified by MS analysis for samples prepared using the combined Protein A depletion and native digestion method and the modified EasyPep sample preparation method.

FIG. 13 is a Venn diagram that illustrates the number of HCPs identified in samples prepared by native digestion preparation, combined Protein A depletion and native digestion, and modified EasyPep sample preparation methods.

FIG. 14 is a schematic diagram comparing the workflows for a modified sample preparation method including separate digestion of supernatant and from beads sample and a modified sample preparation method including combined digestion and clean-up of supernatant and beads.

FIG. 15 is a Venn diagram that illustrates the number of HCPs identified by MS analysis in samples prepared using a supernatant separate digestion method and a combined digestion of supernatant and beads method.

FIG. 16 is a Venn diagram that illustrates the number of HCPs identified in three distinct samples by MS: supernatant separate digestion sample, beads separate digestion sample, and the combined digestion of supernatant and bead sample.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

SEQ ID NOs: 1-116 are amino acid sequences of native HCP peptides.

SEQ ID NOs: 117-232 are amino acid sequences of labeled internal standards for the HCP peptides of SEQ ID NOs: 1-116, respectively.

### DETAILED DESCRIPTION

Provided herein are methods for detecting HCPs in a biological sample, such as an antibody preparation produced in a cell culture system. The disclosed methods utilize MS for detecting HCPs. Antibody preparations typically include large quantities of antibody (such as 10-100 mg antibody) and very low amounts of HCPs. The methods provided herein utilize a modified sample preparation method that removes a large proportion of the antibody, while retaining the HCPs. As demonstrated herein, this reduces dynamic range and results in improved detection of HCPs.

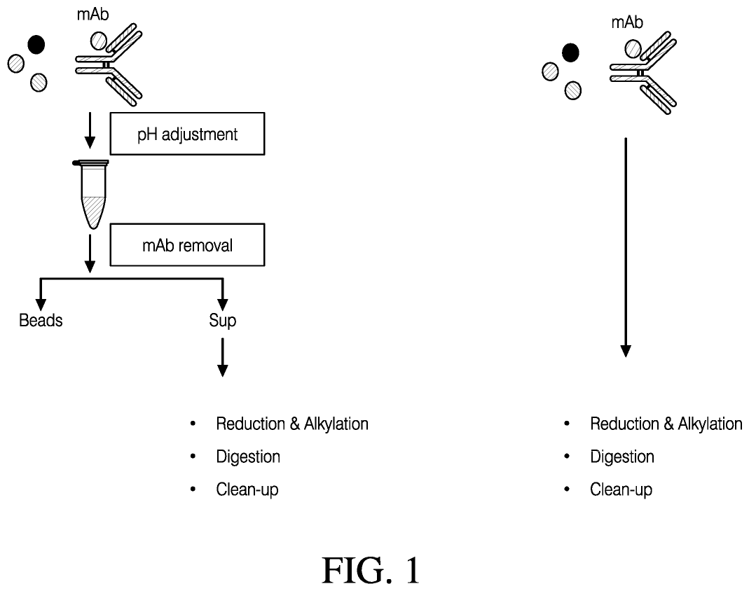

The provided methods include removing antibody from the sample utilizing magnetic beads coated with an alkali resistant immunoglobulin binding protein. During incubation with the beads, the pH is an alkaline pH (FIG. 1). At neutral pH some HCPs interact with the antibody via ionic interactions because the antibody is neutral or cationic, and the majority of HCPs are neutral or anionic. By utilizing an alkaline pH, the antibody is neutral or anionic, which reduces the electrostatic attraction between the antibody and HCPs, causing the HCPs to stay in solution or weakly bound to the antibody. The HCPs in solution can then be detected utilizing mass spectrometry methods with high specificity.

### I. Methods of Detecting One or More Target Proteins

Disclosed herein are methods for detecting one or more target proteins in a biological sample, such as detecting host cell proteins in a recombinant biopharmaceutical product or preparation. In particular examples, the biological sample includes an antibody, for example an antibody produced in a host cell system, and the target proteins include one or more host cell proteins in the sample. In some examples, the one or more target proteins are detected, and in some examples, quantified, using mass spectrometry methods.

In some aspects, the methods include contacting the biological sample with a solid support including an immunoglobulin binding protein, wherein the contacting is carried out at a pH of about 8-10, to produce a sample including the antibody or antibody fragment bound to the solid support. The method further includes a partial enzymatic digestion of the antibody or antibody fragment and target proteins, followed by separating the sample including the antibody or antibody fragment bound to the solid support into fractions including a first fraction including the antibody or antibody fragment bound to the solid support, and a second fraction including a supernatant including the one or more target proteins. The supernatant including the one or more target proteins is processed for analysis and the one or more target proteins are detected.

In some aspects of the methods, the solid support is a bead. In some examples, the bead is a magnetic bead. The solid support includes (for example, is coated with) an immunoglobulin binding protein. Examples of immunoglobulin binding proteins include Protein A, Protein G, Protein A/G, or Protein L. In particular examples, the immunoglobulin binding protein is Protein A. In further examples, the immunoglobulin binding protein is an alkali stable immunoglobulin binding protein, such as alkali stable Protein A. In additional examples, the solid support including the immunoglobulin binding protein has high-capacity binding; however, high-capacity binding is not required for the disclosed methods. For example, binding capacity can be selected based on the starting antibody amount present in the sample. In one example the solid support includes high-capacity immunoglobulin binding protein capable of binding at least 40 mg of immunoglobulin/mL. In a specific, non-limiting example, the solid support is an alkali resistant high capacity Protein A magnetic bead (such as Pierce™ High Capacity Protein A MagBeads, alkali stable, catalog number A53036).

Aspects of the disclosed methods include binding of antibody in the sample to the solid support at an alkaline pH. In some aspects, contacting the biological sample with a solid support including an immunoglobulin binding protein is carried out at a pH of about 8-10, for example in the presence of a buffer of an appropriate pH and/or buffering capacity. In some examples, the pH is about 8 to about 9, about 8.5 to about 9.5, or about 9 to about 10. In particular examples, the pH is about 9.0, about 9.1, about 9.2, about 9.3, about 9.4, about 9.5, about 9.6, about 9.7, about 9.8, about 9.9, or about 10.0. In some examples, the biological sample is contacted with a solid support including an immunoglobulin binding protein in the presence of triethylammonium bicarbonate (TEAB). In other examples, the buffer may be an ammonium bicarbonate, ammonium hydroxide, or carbonate/bicarbonate buffer. One of ordinary skill in the art can select suitable buffers for use in the disclosed methods.

In some aspects, the methods include incubating the sample with the solid support including an immunoglobulin binding protein at an alkaline pH for about 5 minutes to 2 hours (such as about 5-30 minutes, about 15-60 minutes, about 45/90 minutes, or about 1-2 hours). In one example, the sample is incubated with the solid support including an immunoglobulin binding protein at an alkaline pH for 1 hour at room temperature. In some examples, the incubation is carried out with mixing, for example with vortexing.

The method also includes contacting the sample including the antibody or antibody fragment bound to the solid support with a protease agent to produce partially digested antibody and target proteins. In one example, the sample including the antibody or antibody fragment bound to the solid support is contacted with the protease agent for about 15 minutes to 3 hours (such as about 15-45 minutes, about 30-60 minutes, about 45-90 minutes, about 1-2 hours, or about 2-3 hours). In one example, the sample including the antibody or antibody fragment bound to the solid support is contacted with the protease agent for about 1 hour at 37° C. In some examples, the protease agent includes one or more of trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin. In one specific example, the protease agent is trypsin.

The method further includes separating the sample including the antibody or antibody fragment bound to the solid support into fractions including a first fraction including the antibody or antibody fragment bound to the solid support, and a second fraction including a supernatant including the one or more target proteins. In some examples, the separating includes centrifugation, for example, when the solid support is a bead. In other examples, the separating includes exposing the sample to a magnet, for example, when the solid support is a magnetic bead. The antibody remains primarily bound to the solid support and the one or more target proteins remain primarily in the supernatant.

The method can include combining the first fraction and the second fraction. The disclosed methods include processing the supernatant including the one or more target proteins for analysis and detection of the target proteins. In some aspects, the methods include reducing and alkylating the one or more target proteins and digesting the reduced and alkylated target proteins with one or more protease agents to produce digested peptides. In some examples, the one or more protease agents includes one or more of trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin. In one specific example, the protease agent is trypsin.

In some aspects, the methods further include purifying the digested peptides, for example, prior to analysis. In some examples, the digested peptides are purified using an ion exchange material, such as a hydrophobic, polymer-based cation exchange material. The purifying may include contacting the digested peptides with a hydrophobic, polymer-based cation exchange material under acidic conditions, such that the digested peptides bind to the cation exchange material, washing the cation exchange material with a first acidic solution, such that the digested peptides are retained on the cation exchange material and hydrophilic contaminants are removed from the cation exchange material, washing the cation exchange material with a second acidic solution, such that the digested peptides are retained on the cation exchange material and monovalent cation and/or hydrophobic contaminants are removed from the cation exchange material, and eluting the retained digested peptides form the cation exchange material with an alkaline or neutral solution. In some examples, the digested peptides are purified using an EasyPep™ MS Sample Prep Kit (e.g., Thermo Fisher Catalog number A45734). Alternative purification methods can be used with the workflows provided herein such as ion exchange, hydrophobic, and polymer-based cation exchange.

The methods further include detecting the one or more target proteins. In some aspects, detecting the one or more target proteins includes assaying the digested peptides via mass spectrometry (MS) to determine the presence of at least one peptide from one or more target proteins. In some examples, the mass spectrometry includes liquid chromatography-mass spectrometry (LC-MS). In some examples, the detecting includes data dependent analysis (DDA), data independent analysis (DIA), parallel reaction monitoring (PRM), selected reaction monitoring (SRM), or other targeted MS acquisition methods. In some aspects, detecting the one or more target proteins includes assaying the digested peptides via ELISA methods to determine the presence of at least one peptide from one or more target proteins.

In some aspects, the MS methods includes adding one or more internal standard peptides of a known amount to the digested peptides prior to mass spectrometry. The one or more internal standard peptides have the same amino acid sequence as at least one target peptide, and are detectably labeled. The quantity of the target peptide is determined by comparison to the internal standard. In some aspects, the detectable label is linked to or incorporated in the internal standard peptide. In some examples, the label is a mass-altering label. The mass-altering label may include an isotope (such as ¹³C, ¹⁵N, ²H, and/or ¹⁸O), such as an amino acid including an isotopic label. In other examples, the mass-altering label includes a modified amino acid, such as carbamidomethyl-cysteine (CAM-C). In some examples, the quantity of one or more target proteins is determined by comparing an amount of a target peptide in the sample to the amount of the same target peptide in a control sample.

In some aspects, the target peptide includes or consists of the amino acid sequence of any one of SEQ ID NOs: 1-116. In other aspects, the detectably labeled internal standard peptide includes the amino acid sequence of any one of SEQ ID NOs: 1-116. In particular examples, the detectably labeled internal standard peptide includes or consists of the amino acid sequence of any one of SEQ ID NOs: 117-232. In other examples, the target peptides include the amino acid sequence of each of SEQ ID NOs: 1, 4, 6, 7, 8, 11, 13, 14, 15, 17, 18, 19, 20, 22, 23, 24, 26, 27, 29, 30, 31, 32, 33, 34, 35, 37, 38, 40, 44, 45, 46, 48, 49, 51, 52, 53, 54, 56, 59, 61, 64, 65, 66, 67, 70, 71, 76, 77, 81, 82, 83, 84, 85, 87, 89, 90, 94, 96, 98, 99, 101, 102, 106, 108, 110, 111, 112, 113, 115, and 116. In other examples, a set of detectably labeled internal standard peptides include each of SEQ ID NOs: 117, 120, 122, 123, 124, 127, 129, 130, 131, 133, 134, 135, 136, 138, 139, 140, 142, 143, 145, 146, 147, 148, 149, 150, 151, 153, 154, 156, 160, 161, 162, 164, 165, 167, 168, 169, 170, 172, 175, 177, 180, 181, 182, 183, 186, 187, 192, 193, 197, 198, 199, 200, 201, 203, 205, 206, 210, 212, 214, 215, 217, 218, 222, 224, 226, 227, 228, 229, 231, and 232.

In some aspects the sample is a recombinant biopharmaceutical product or preparation, such as a recombinant biopharmaceutical product generated in a host cell culture, for example, from a mammalian cell line, such as CHO cells, mouse myeloma-derived NS0 or Sp2/0 cells, human embryonic kidney cells (HEK293), or human embryonic retinoblast-derived PER.C6 cells. In some examples, the sample is a recombinant antibody preparation. In one example, the sample is a trastuzumab (Herceptin) preparation. In some examples, the sample is a liquid sample. In other examples, the sample is a dry (e.g., lyophilized) sample, which may be reconstituted prior to use, for example by addition of water or a buffer.

In some aspects the one or more target proteins are one or more host cell proteins. “Host cell proteins” refers to process-related impurities in recombinant biopharmaceutical products or preparations (for example, therapeutic antibodies) that are generated by the host cell or organism and are typically present at low levels in the product or preparation. In some aspects, the one or more host cell proteins are critical HCPs. Critical HCPs (also referred to as high-risk HCPs) are potentially problematic HCPs and can include those that are immunogenic, biologically active, or enzymatically active, for example, with the potential to degrade either product molecules or excipients used in a recombinant therapeutic formulation. Some critical HCPs have also been shown to be difficult to remove by purification. Exemplary critical host cell proteins are provided in Table 1. In some examples, the disclosed methods detect at least 20 critical HCPs (such as at least 20, at least 21, at least 22, at least 23, at least 24, at least 25, at least 26, at least 27, at least 28, at least 29, or at least 30 critical HCPs) in a sample. In some examples, the disclosed methods also detect the recombinant biopharmaceutical in the sample (such as an antibody heavy chain, light chain, or both).

TABLE 1
Exemplary critical host cell proteins

| Protein | |
|---|---|
| Accession No. | Protein |
| GI18R9 | 78 kDa glucose regulated protein(GRP78, BiP) |
| G3I0W1 | Alpha-enolase (2-phospho-D-glycerate hydro-lyase) |
| G3I5A4 | Annexin A5 (ANXA5) |
| G3GTT2 | C-C motif chemokine (CCL) |
| A4URF0 | C-X-C motif chemokine 3 (CXCL3) |
| A0A061IFE2 | Carboxylesterase 1-like protein, Liver (CES1) |
| A0A061I7X9 | Carboxylesterase B-1-like protein, Liver (CES-B1L) |
| G3HR95 | Carboxypeptidase D (Cpd) |
| G3H0L9 | Cathepsin B (CatB) |
| G3I4W7 | Cathepsin D (CatD) |
| G3INC5 | Cathepsin L (CatL) |
| Q9EPP7 | Cathepsin Z (CatZ) |
| G3HNJ3 | Clusterin (CLU) |
| G3I3Y6 | Glutathione S-transferase P 1 (GSTP1) |
| G3H6V7 | Lipoprotein Lipase (LPL) |
| G3HQY6 | Lysosomal Acid Lipase (LAL) |
| G3HKV9 | Lysosomal Phospholipase A2 (LPLA2) |
| G3HRK9 | Matrix metalloproteinase-19 (MMP-19) |
| P14851 | Peptidyl-prolyl cis-trans isomerase A (PPIA) |
| Q9JKY1 | Peroxiredoxin-1 (PRDX1) |
| G3I6T1 | Phospholipase B-like 2 (PLBL2) |
| G3IIE7 | Procollagen-lysine 2-oxoglutarate 5-deoxygenase_1 |
| | (PLOD1) |
| Q8R4U2 | Protein disulfide isomerase (PDI) |
| G3HC31 | Protein S100-A6 (S10A6) |
| G3H3Q1 | Pyruvate Kinase (PK) |
| G3IBF4 | Serine protease (HTRA1) |
| A0A3L7HS03 | Sialate o-acetylesterase (SIAE) |
| G3IA12 | Transforming Growth Factor-b1 (TGF-β1) |

### II. Compositions and Kits

Provided herein are compositions and kits for detecting one or more host cell proteins in a sample. In some aspects, the compositions and kits include one or more detectably labeled peptides derived from HCPs. The detectably labeled peptides can be used as internal control peptides for detection of HCPs by mass spectrometry, including, but not limited to the methods described herein.

In some aspects the compositions include detectably labeled peptides including or consisting of the amino acid sequence of any one of SEQ ID NOs: 1-116. In some aspects, the detectable label is a mass-altering label such as include an isotope (such as ¹³C, ¹⁵N, ²H, and/or ¹⁸O), such as an amino acid include an isotopic label. In other examples, the mass-altering label includes a modified amino acid, such as carbamidomethyl-cysteine (CAM-C). In particular examples, the detectably labeled peptides include or consist of one or more of SEQ ID NOs: 117-232.

Also provided are sets of peptides, such as 1-25, 20-50, 40-75, 60-100, or 80-116 detectably labeled peptides including or consisting of the amino acid sequence of any one of SEQ ID NOs: 1-232. In one example, a set of peptides includes detectably labeled peptides including the amino acid sequence of each of SEQ ID NOs: 1, 4, 6, 7, 8, 11, 13, 14, 15, 17, 18, 19, 20, 22, 23, 24, 26, 27, 29, 30, 31, 32, 33, 34, 35, 37, 38, 40, 44, 45, 46, 48, 49, 51, 52, 53, 54, 56, 59, 61, 64, 65, 66, 67, 70, 71, 76, 77, 81, 82, 83, 84, 85, 87, 89, 90, 94, 96, 98, 99, 101, 102, 106, 108, 110, 111, 112, 113, 115, and 116. In other examples, a set of detectably labeled peptides includes each of SEQ ID NOs: 117, 120, 122, 123, 124, 127, 129, 130, 131, 133, 134, 135, 136, 138, 139, 140, 142, 143, 145, 146, 147, 148, 149, 150, 151, 153, 154, 156, 160, 161, 162, 164, 165, 167, 168, 169, 170, 172, 175, 177, 180, 181, 182, 183, 186, 187, 192, 193, 197, 198, 199, 200, 201, 203, 205, 206, 210, 212, 214, 215, 217, 218, 222, 224, 226, 227, 228, 229, 231, and 232.

In some aspects kits including one or more detectably labeled peptides including or consisting of the amino acid sequence of any one of SEQ ID NOs: 1-116 are provided. The kit may include 1-25, 20-50, 40-75, 60-100, or 80-116 detectably labeled peptides including or consisting of the amino acid sequence of any one of SEQ ID NOs: 1-116. In one example, the kit includes 70 detectably labeled peptides.

In some aspects, the kit includes one or more detectably labeled peptides where the detectable label is a mass-altering label such as include an isotope (such as ¹³C, ¹⁵N, ²H, and/or ¹⁸O), such as an amino acid include an isotopic label. In other examples, the mass-altering label includes a modified amino acid, such as carbamidomethyl-cysteine (CAM-C). In particular examples, the one or more detectably labeled peptides include or consist of one or more of SEQ ID NOs: 117-232.

In some examples, the kit includes detectably labeled peptides including the amino acid sequence of each of SEQ ID NOs: 1, 4, 6, 7, 8, 11, 13, 14, 15, 17, 18, 19, 20, 22, 23, 24, 26, 27, 29, 30, 31, 32, 33, 34, 35, 37, 38, 40, 44, 45, 46, 48, 49, 51, 52, 53, 54, 56, 59, 61, 64, 65, 66, 67, 70, 71, 76, 77, 81, 82, 83, 84, 85, 87, 89, 90, 94, 96, 98, 99, 101, 102, 106, 108, 110, 111, 112, 113, 115, and 116. In other examples, the kit includes detectably labeled peptides including or consisting of each of SEQ ID NOs: 117, 120, 122, 123, 124, 127, 129, 130, 131, 133, 134, 135, 136, 138, 139, 140, 142, 143, 145, 146, 147, 148, 149, 150, 151, 153, 154, 156, 160, 161, 162, 164, 165, 167, 168, 169, 170, 172, 175, 177, 180, 181, 182, 183, 186, 187, 192, 193, 197, 198, 199, 200, 201, 203, 205, 206, 210, 212, 214, 215, 217, 218, 222, 224, 226, 227, 228, 229, 231, and 232.

In additional aspects, the kit includes one or more additional reagents for carrying out sample preparation and/or mass spectrometry methods, including one or more buffers (such as lysis buffer or wash buffers), reduction solution, alkylation solution, stop solution, enzymes, purification columns, and the like. In some examples, the kit includes one or more protease agents, such as one or more of trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, and thermolysin. In additional examples, the kit includes a solid support, such as beads (for example, magnetic beads) coated with an immunoglobulin binding protein (for example, Protein A magnetic beads). In further examples, the kit may also include one or more controls, such as one or more positive control samples. In some examples, the kit includes one or more dry (e.g., lyophilized) reagents or samples, which may be reconstituted prior to use (for example, using water or a buffer). In further examples, the kit may include one or more tubes, sample plates (such as 96 well plates), or other receptacles.

### EXAMPLES

The following examples are provided to illustrate particular features of certain aspects of the disclosure, but the scope of the claims should not be limited to those features exemplified.

### Example 1

### Improved Methods for Detecting Host Cell Proteins in an Antibody Preparation

Experiments were performed to evaluate a modified sample preparation method compared with the regular EasyPep maxi method (see, e.g., FIG. 1). The two methods were evaluated to determine which method is better to detect number of identified HCP proteins and number of quantifiable HCP peptides by performing LC/MS DDA and PRM data acquisition using Herceptin monoclonal antibodies.

Samples were prepared for detection of HCPs by mass spectrometry, including pH adjustment and antibody removal. A monoclonal antibody preparation (200 μL of 5 mg/mL Herceptin antibody stock) was mixed with 22 μL 1 M triethylammonium bicarbonate (TEAB). This was added to 30 μL high capacity protein A MagBeads, alkali stable (Pierce™ catalog number A53038) that were washed three times with 100 mM TEAB. The mixture was vortexed for 1 hour at room temperature. The sample was split into two parts. The first half of sample was incubated with 1:50 ratio of trypsin to sample for 1 hr at 37° C. The magnetic beads were collected and the supernatant was transferred to a new tube. The beads were washed with 280 μL 100 mM TEAB and collected again. The supernatant was combined with the previously collected supernatant (Ab-removed sample). A control antibody sample was prepared by mixing 200 μL of the antibody stock with 250 μL LCMS grade water and 50 μL 1 M TEAB.

The samples were then processed using the EasyPep™ Maxi MS sample kit (Thermo Fisher Scientific, catalog number A45734). Briefly, 0.25 mL of reduction solution and 0.25 mL of alkylation solution were added to each sample and incubated for 10 minutes at 95° C. with 600 rpm mixing. After cooling to room temperature, 25 μL of trypsin (2 μg/μL) was added and the samples were incubated at 37° C. for 2.5 hour at 1400 rpm. The reaction was stopped by adding 0.25 mL of stop solution and pH was checked (should be pH<3). Peptides were cleaned up by adding each sample (~1.25 mL) to a peptide clean-up column and centrifuging at 2000×g for 2 minutes. The column was washed with 3 mL Wash Solution A, followed by 2-3 washes with Wash Solution B, with centrifugation for 2 minutes at 2000×g for each wash. Then, 3 mL of Elution Solution was added to the column and centrifuged for 2 minutes at 2000×g. The eluted peptides were transferred to low protein binding tubes and dried using a vacuum centrifuge. The sample was resuspended in 250-500 μL 0.1% formic acid in water and peptide concentration was determined using Pierce™ quantitative peptide assay (catalog number 23275).

Samples were prepared for PRM analysis by mixing 1 μL (0.5 μg/μL peptide sample) with 2.8 μL (71 fmol/μL labeled internal standard peptides) and 0.2 μL peptide diluent. For discovery and targeted PRM analysis, the HCP digest samples with heavy peptide mixture were analyzed by a Thermo Scientific™ Dionex™ UltiMate™ 3000 RSLCnano System coupled to a Thermo Scientific™ Q Exactive™ plus or Q Exactive™ HF Hybrid Quadrupole-Orbitrap Mass Spectrometer.

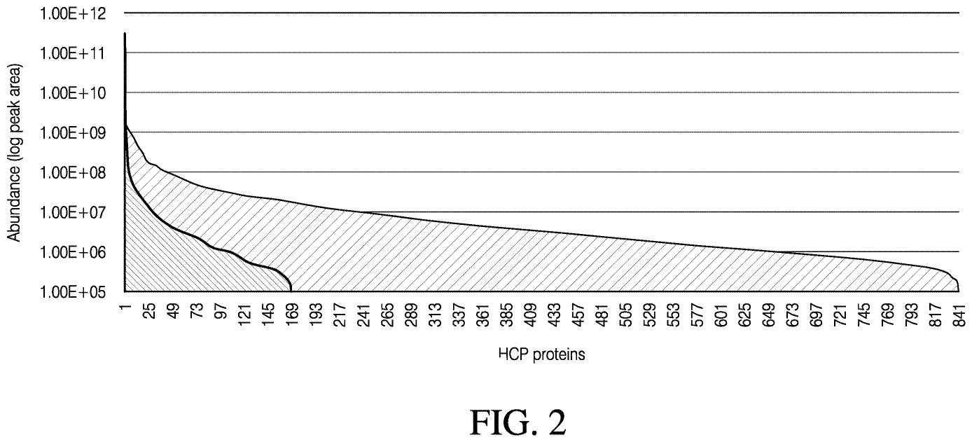

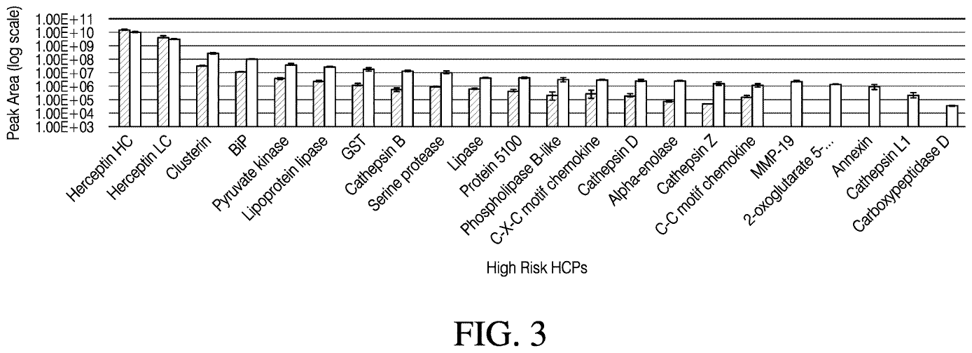

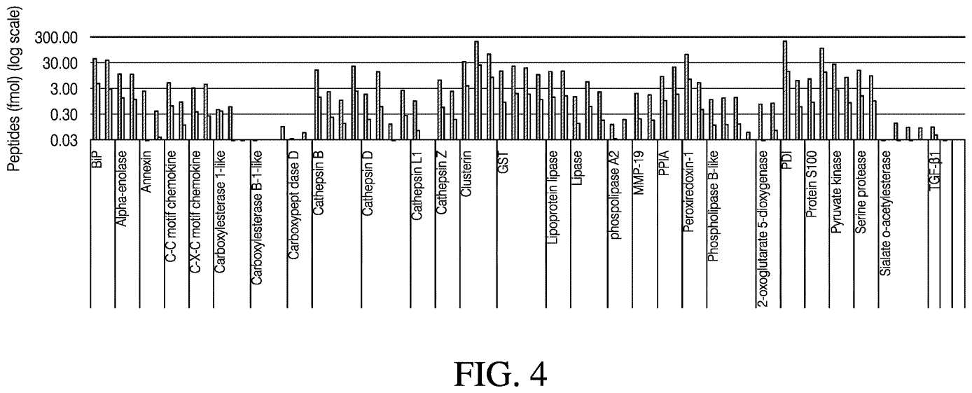

Using the modified method described above, 861 HCPs were identified in the sample, whereas 173 HCPs were identified with the regular EasyPep method (FIG. 2). This is almost one order of magnitude reduction of dynamic range with the modified method. In addition, the modified methods identified 22 “critical” HCP proteins, compared to 17 in the regular EasyPep method (FIG. 3). Ten more peptides were quantified in the modified method compared to the regular method (FIG. 4).

### Example 2

### Multiplex Quantitation of Critical HCPs Using Targeted Mass Spectrometry Assay Peptide Panel

### Materials and Methods

The EasyPep™ MS sample prep kit (Thermo Fisher Cat. No. A40006) was used to denature, reduce, alkylate, digest, and clean up the HCP sample protein digest (prepared as described in Example 1). Traditional sample preparation method includes Guanidine-HCl based denaturation, reduction, alkylation, and trypsin digestion. The modified traditional sample preparation method is the same as the traditional method except that desalting step was performed using Pierce™ polyacrylamide spin desalting column (Cat. No. 89849).

200 fmol of the critical HCPs AQUA heavy peptide mixture was spiked in 0.5 μg of each digest sample or Pierce™ 6 Protein Digest (Cat. No. 88342) and Pierce™ Peptide Retention Time Calibration Mixture (Cat. No. 88320). For discovery and targeted PRM analysis, the HCP digest samples with heavy peptide mixture were analyzed by a Thermo Scientific™ Dionex™ UltiMate™ 3000 RSLCnano System coupled to a Thermo Scientific™ Q Exactive™ plus or Q Exactive™ HF Hybrid Quadrupole-Orbitrap Mass Spectrometer.

Proteome Discoverer software was used to search discovery MS data. For targeted data analysis, Skyline software (University of Washington) was used.

### Results

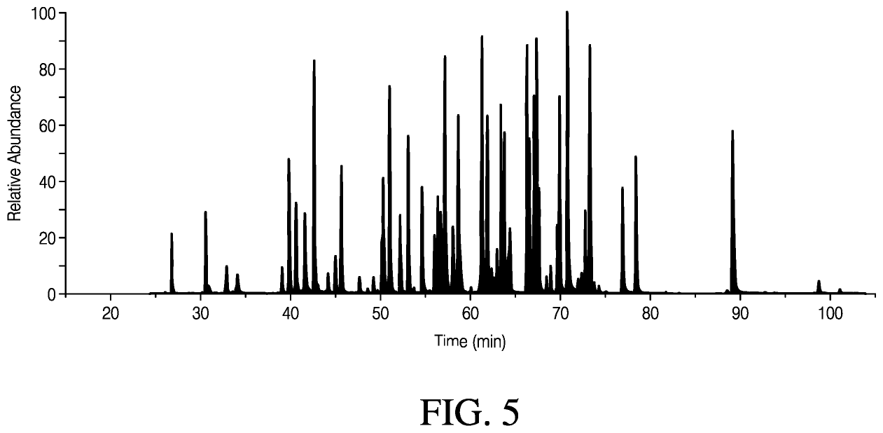

The CHO HCP standard proteins were digested with two different sample preparation methods. Proteome Discoverer data processing generated >3000 HCPs and >20,000 peptides. An HCP peptide spectral library was created in Skyline software and selected 28 critical HCPs. Five unique peptides per protein were selected from spectral library dataset and 116 crude peptides were synthesized (Table 2). For the final assay development, the 70 best-performing peptides (indicated in Table 2 with *) from 28 critical HCPs were selected for the AQUA grade heavy peptide synthesis (FIG. 5). The critical HCPs heavy AQUA peptide mixture was created by mixing equal amounts of each peptide. Then, 200 fmol of each peptide in the mixture was spiked into 2 μmol of a relevant matrix of six proteins digest and analyzed by DDA and PRM method (FIG. 5). All 70 AQUA heavy peptides were verified by both DDA and PRM analysis.

TABLE 2
HCP peptides

| | | SEQ | | SEQ |
|---|---|---|---|---|
| | Native Peptide | ID | | ID |
| Target ID | Sequence | NO: | Internal Standard Peptide Sequence | NO: |
| G3I8R9_1* | VEIIANDQGNR | 1 | VEIIANDQGN[R(¹³C6; ¹⁵N4)] | 117 |
| G3I8R9_2 | ITPSYVAFTPEGER | 2 | ITPSYVAFTPEGE[R(¹³C6; ¹⁵N4)] | 118 |
| G3I8R9_3 | TWNDPSVQQDIK | 3 | TWNDPSVQQDI[K(¹³C6; ¹⁵N2)] | 119 |
| G3I8R9_4* | VTHAVVTVPAYFNDAQR | 4 | VTHAVVTVPAYFNDAQ[R(¹³C6; ¹⁵N4)] | 120 |
| G3I8R9_5 | SQIFSTASDNQPTVTIK | 5 | SQIFSTASDNQPTVTI[K(¹³C6; ¹⁵N2)] | 121 |
| G3I0W1_1* | IGAEVYHNLK | 6 | IGAEVYHNL[K(¹³C6; ¹⁵N2)] | 122 |
| G3I0W1_2* | S[C(CAM)]N[C(CAM)]LLLK | 7 | S[C(CAM)]N[C(CAM)]LLL[K(¹³C6; ¹⁵N2)] | 123 |
| G3I5A4_1* | GTVTAFPGFDSR | 8 | GTVTAFPGFDS[R(¹³C6; ¹⁵N4)] | 124 |
| G3I5A4_2 | GLGTDEESILTLLTSR | 9 | GLGTDEESILTLLTS[R(¹³C6; ¹⁵N4)] | 125 |
| G3I5A4_3 | LYDAYELK | 10 | LYDAYEL[K(¹³C6; ¹⁵N2)] | 126 |
| G3I5A4_4* | VLTEIIASR | 11 | VLTEIIAS[R(¹³C6; ¹⁵N4)] | 127 |
| G3I5A4_5 | FITIFGTR | 12 | FITIFGT[R(¹³C6; ¹⁵N4)] | 128 |
| G3GTT2_1* | EAVIFITK | 13 | EAVIFIT[K(¹³C6; ¹⁵N2)] | 129 |
| G3GTT2_2* | QDWVQTYTK | 14 | QDWVQTYT[K(¹³C6; ¹⁵N2)] | 130 |
| A4URF0_1* | IDFEDIQSLK | 15 | IDFEDIQSL[K(¹³C6; ¹⁵N2)] | 131 |
| A4URF0_2 | VTPPGPH[C(CAM)]TQTE | 16 | VTPPGPH[C(CAM)]TQTEVIATL[K(¹³C6; | 132 |
| | VIATLK | | ¹⁵N2)] | |
| A4URF0_3* | DGQEV[C(CAM)]LDPEGPR | 17 | DGQEV[C(CAM)]LDPEGP[R(¹³C6; ¹⁵N4)] | 133 |
| A0A061IFE2_1* | FGALDFLGDPR | 18 | FGALDFLGDP[R(¹³C6; ¹⁵N4)] | 134 |
| A0A061IFE2_2* | AISESGVILVPGLFTK | 19 | AISESGVILVPGLFT[K(¹³C6; ¹⁵N2)] | 135 |
| A0A061IFE2_3* | ENHAFVPTVLDGVLLPK | 20 | ENHAFVPTVLDGVLLP[K(¹³C6; ¹⁵N2)] | 136 |
| A0A061IFE2_4 | APEEILAEK | 21 | APEEILAE[K(¹³C6; ¹⁵N2)] | 137 |
| A0A061I7X9_1* | TTTSAVIVH[C(CAM)]LR | 22 | TTTSAVIVH [C(CAM)]L[R(¹³C6; ¹⁵N4)] | 138 |
| A0A061I7X9_2* | FWANFAR | 23 | FWANFA[R(¹³C6; ¹⁵N4)] | 139 |
| A0A061I7X9_3* | NGNPNGEGLPHWPEYDQK | 24 | NGNPNGEGLPHWPEYDQ[K(¹³C6; ¹⁵N2)] | 140 |
| G3HR95_1 | ESLITLIEK | 25 | ESLITLIE[K(¹³C6; ¹⁵N2)] | 141 |
| G3HR95_2* | NFGTDPEVTDLVR | 26 | NFGTDPEVTDLV[R(¹³C6; ¹⁵N4)] | 142 |
| G3HR95_3* | SQEGDSVSVVGR | 27 | SQEGDSVSVVG[R(¹³C6; ¹⁵N4)] | 143 |
| G3HR95_4 | LLVPGTYK | 28 | LLVPGTY[K(¹³C6; ¹⁵N2)] | 144 |
| G3H0L9_1* | EQWSN[C(CAM)]PTIK | 29 | EQWSN[C(CAM)]PTI[K(¹³C6; ¹⁵N2)] | 145 |
| G3H0L9_2* | S[C(CAM)]EAGYSPSYK | 30 | S[C(CAM)]EAGYSPSY[K(¹³C6; ¹⁵N2)] | 146 |
| G3H0L9_3* | HYGYTSYSVSNNEK | 31 | HYGYTSYSVSNNE[K(¹³C6; ¹⁵N2)] | 147 |
| G3H0L9_4* | GEDH[C(CAM)]GIESEIV | 32 | GEDH[C(CAM)]GIESEIVAGIP[R(¹³C6; | 148 |
| | AGIPR | | ¹⁵N4)] | |
| G3I4W7_1* | GPVSELLK | 33 | GPVSELL[K(¹³C6; ¹⁵N2)] | 149 |
| G3I4W7_2* | LLDIA[C(CAM)]WIHHK | 34 | LLDIA[C(CAM)]WIHH[K(¹³C6; ¹⁵N2)] | 150 |
| G3I4W7_3* | NIFSFFLNR | 35 | NIFSFFLN[R(¹³C6; ¹⁵N4)] | 151 |
| G3I4W7_4 | GG[C(CAM)]EAIVDTGTS | 36 | GG[C(CAM)]EAIVDTGTSLLVGPVDEV[K(¹³C6; | 152 |
| | LLVGPVDEVK | | ¹⁵N2)] | |
| G3I4W7_5* | VSSLPSVTLK | 37 | VSSLPSVTL[K(¹³C6; ¹⁵N2)] | 153 |
| G3INC5_1* | LYGTNEEEWR | 38 | LYGTNEEEW[R(¹³C6; ¹⁵N4)] | 154 |
| G3INC5_2 | GLDSEESYPYEAK | 39 | GLDSEESYPYEA[K(¹³C6; ¹⁵N2)] | 155 |
| G3INC5_3* | YKPEFAAANDTGYVDIPQLEK | 40 | YKPEFAAANDTGYVDIPQLE[K(¹³C6; ¹⁵N2)] | 156 |
| Q9EPP7_1 | TYPRPHEYLSPSDIPK | 41 | TYPRPHEYLSPSDIP[K(¹³C6; ¹⁵N2)] | 157 |
| Q9EPP7_2 | GVNYASITR | 42 | GVNYASIT[R(¹³C6; ¹⁵N4)] | 158 |
| Q9EPP7_3 | FNQ[C(CAM)]GT[C(CAM)] | 43 | FNQ[C(CAM)]GT[C(CAM)]TEF[K(¹³C6; | 159 |
| | TEFK | | ¹⁵N2)] | |
| Q9EPP7_4* | VGDYGSLSGR | 44 | VGDYGSLSG[R(¹³C6; ¹⁵N4)] | 160 |
| Q9EPP7_5* | NSWGEPWGER | 45 | NSWGEPWGE[R(¹³C6; ¹⁵N4)] | 161 |
| G3HNJ3_1* | EIQNAVQGVK | 46 | EIQNAVQGV[K(¹³C6; ¹⁵N2)] | 162 |
| G3HNJ3_2 | RPHFLYPK | 47 | RPHFLYP[K(¹³C6; ¹⁵N2)] | 163 |
| G3HNJ3_3* | LTQQYNELLHSLQTK | 48 | LTQQYNELLHSLQT[K(¹³C6; ¹⁵N2)] | 164 |
| G3HNJ3_4* | VSTVTTHSNNSEEPSR | 49 | VSTVTTHSNNSEEPS[R(¹³C6; ¹⁵N4)] | 165 |
| G3HNJ3_5 | LFDSDPITVVLPEEVSK | 50 | LFDSDPITVVLPEEVS[K(¹³C6; ¹⁵N2)] | 166 |
| G3I3Y6_1* | ILLADQGQSWK | 51 | ILLADQGQSW[K(¹³C6; ¹⁵N2)] | 167 |
| G3I3Y6_2* | ST[C(CAM)]LYGQLPK | 52 | ST[C(CAM)]LYGQLP[K(¹³C6; ¹⁵N2)] | 168 |
| G3I3Y6_3* | FEDGDLTLYQSNAILR | 53 | FEDGDLTLYQSNAIL[R(¹³C6; ¹⁵N4)] | 169 |
| G3I3Y6_4* | YITLIYTK | 54 | YITLIYT[K(¹³C6; ¹⁵N2)] | 170 |
| G3I3Y6_5 | ALPGHLKPFETLLSQNQGGK | 55 | ALPGHLKPFETLLSQNQGG[K(¹³C6; ¹⁵N2)] | 171 |
| G3H6V7_1* | DFTDIESK | 56 | DFTDIES[K(¹³C6; ¹⁵N2)] | 172 |
| G3H6V7_2 | AQQHYPVSAGYTK | 57 | AQQHYPVSAGYT[K(¹³C6; ¹⁵N2)] | 173 |
| G3H6V7_3 | ITGLDPAGPNFEYAEAPSR | 58 | ITGLDPAGPNFEYAEAPS[R(¹³C6; ¹⁵N4)] | 174 |
| G3H6V7_4* | GLGDVDQLVK | 59 | GLGDVDQLV[K(¹³C6; ¹⁵N2)] | 175 |
| G3H6V7_5 | SIHLFIDSLLNEENPSK | 60 | SIHLFIDSLLNEENPS[K(¹³C6; ¹⁵N2)] | 176 |
| G3HQY6_1* | GNTWSLK | 61 | GNTWSL[K(¹³C6; ¹⁵N2)] | 177 |
| G3HQY6_2 | WPEVIIEDLFGHK | 62 | WPEVIIEDLFGH[K(¹³C6; ¹⁵N2)] | 178 |
| G3HQY6_3 | WLSTHV[C(CAM)]NR | 63 | WLSTHV[C(CAM)]N[R(¹³C6; ¹⁵N4)] | 179 |
| G3HQY6_4* | VNVYTSHSPAGTSVQNLR | 64 | VNVYTSHSPAGTSVQNL[R(¹³C6; ¹⁵N4)] | 180 |
| G3HQY6_5* | HWGQIAK | 65 | HWGQIA[K(¹³C6; ¹⁵N2)] | 181 |
| G3HKV9_1* | HPPVVLVPGDLGNQLEAK | 66 | HPPVVLVPGDLGNQLEA[K(¹³C6; ¹⁵N2)] | 182 |
| G3HKV9_2* | ATQFPDGVDVR | 67 | ATQFPDGVDV[R(¹³C6; ¹⁵N4)] | 183 |
| G3HKV9_3 | IPVIGPLK | 68 | IPVIGPL[K(¹³C6; ¹⁵N2)] | 184 |
| G3HKV9_4 | DYHQFFQDIR | 69 | DYHQFFQDI[R(¹³C6; ¹⁵N4)] | 185 |
| G3HRK9_1* | LEDITEALR | 70 | LEDITEAL[R(¹³C6; ¹⁵N4)] | 186 |
| G3HRK9_2* | ILNLPSTLSPSR | 71 | ILNLPSTLSPS[R(¹³C6; ¹⁵N4)] | 187 |
| G3HRK9_3 | GDYVWTVTDSGPGPLFR | 72 | GDYVWTVTDSGPGPLF[R(¹³C6; ¹⁵N4)] | 188 |
| G3HRK9_4 | TGWIHFFK | 73 | TGWIHFF[K(¹³C6; ¹⁵N2)] | 189 |
| G3HRK9_5 | VEPNLDAALYWPVNQK | 74 | VEPNLDAALYWPVNQ[K(¹³C6; ¹⁵N2)] | 190 |
| P14851_1 | VSFELFADK | 75 | VSFELFAD[K(¹³C6; ¹⁵N2)] | 191 |
| P14851_2* | FEDENFILK | 76 | FEDENFIL[K(¹³C6; ¹⁵N2)] | 192 |
| P14851_3* | TEWLDGK | 77 | TEWLDG[K(¹³C6; ¹⁵N2)] | 193 |
| Q9JKY1_1 | IGYPAPNFK | 78 | IGYPAPNF[K(¹³C6; ¹⁵N2)] | 194 |
| Q9JKY1_2 | DI[C(CAM)]LSEYR | 79 | DI[C(CAM)]LSEY[R(¹³C6; ¹⁵N4)] | 195 |
| Q9JKY1_3 | ADEGISFR | 80 | ADEGISF[R(¹³C6; ¹⁵N4)] | 196 |
| Q9JKY1_4* | GLFIIDDK | 81 | GLFIIDD[K(¹³C6; ¹⁵N2)] | 197 |
| Q9JKY1_5* | SVDEILR | 82 | SVDEIL[R(¹³C6; ¹⁵N4)] | 198 |
| G3I6T1_1* | SVLLDAASGQLR | 83 | SVLLDAASGQL[R(¹³C6; ¹⁵N4)] | 199 |
| G3I6T1_2* | LTLLQLK | 84 | LTLLQL[K(¹³C6; ¹⁵N2)] | 200 |
| G3I6T1_3* | LSLGSGS[C(CAM)]SAIIK | 85 | LSLGSGS[C(CAM)]SAII[K(¹³C6; ¹⁵N2)] | 201 |
| G3I6T1_4 | AFIPNGPSPGSR | 86 | AFIPNGPSPGS[R(¹³C6; ¹⁵N4)] | 202 |
| G3I6T1_5* | VTSFSLAK | 87 | VTSFSLA[K(¹³C6; ¹⁵N2)] | 203 |
| G3IIE7_1 | SAQFFNYK | 88 | SAQFFNY[K(¹³C6; ¹⁵N2)] | 204 |
| G3IIE7_2* | FLGSGGFIGYAPNLNK | 89 | FLGSGGFIGYAPNLN[K(¹³C6; ¹⁵N2)] | 205 |
| G3IIE7_3* | NLAYDTLPVVIHGNGPTK | 90 | NLAYDTLPVVIHGNGPT[K(¹³C6; ¹⁵N2)] | 206 |
| G3IIE7_4 | FLAEHGTEYQSVK | 91 | FLAEHGTEYQSV[K(¹³C6; ¹⁵N2)] | 207 |
| G3IIE7_5 | LLIEQNK | 92 | LLIEQN[K(¹³C6; ¹⁵N2)] | 208 |
| Q8R4U2_1 | VDATEESDLAQQYGVR | 93 | VDATEESDLAQQYGV[R(¹³C6; ¹⁵N4)] | 209 |
| Q8R4U2_2* | HNQLPLVIEFTEQTAPK | 94 | HNQLPLVIEFTEQTAP[K(¹³C6; ¹⁵N2)] | 210 |
| Q8R4U2_3 | ILFIFIDSDHTDNQR | 95 | ILFIFIDSDHTDNQ[R(¹³C6; ¹⁵N4)] | 211 |
| Q8R4U2_4* | YKPESDELTAEK | 96 | YKPESDELTAE[K(¹³C6; ¹⁵N2)] | 212 |
| Q8R4U2_5 | TVIDYNGER | 97 | TVIDYNGE[R(¹³C6; ¹⁵N4)] | 213 |
| G3HC31_1* | LQDAEIAR | 98 | LQDAEIA[R(¹³C6; ¹⁵N4)] | 214 |
| G3HC31_2* | LMDDLDR | 99 | LMDDLD[R(¹³C6; ¹⁵N4)] | 215 |
| G3HC31_3 | DQEVNFQEYVAFLGALA | 100 | DQEVNFQEYVAFLGALALIYNEAL[K(¹³C6; | 216 |
| | LIYNEALK | | ¹⁵N2)] | |
| G3H3Q1_1* | GDLGIEIPAEK | 101 | GDLGIEIPAE[K(¹³C6; ¹⁵N2)] | 217 |
| G3H3Q1_2* | GDYPLEAVR | 102 | GDYPLEAV[R(¹³C6; ¹⁵N4)] | 218 |
| G3H3Q1_3 | APIIAVTR | 103 | APIIAVT[R(¹³C6; ¹⁵N4)] | 219 |
| G3H3Q1_4 | GIFPVL[C(CAM)]K | 104 | GIFPVL[C(CAM)][K(¹³C6; ¹⁵N2)] | 220 |
| G3H3Q1_5 | DAVQDAWAEDVDLR | 105 | DAVQDAWAEDVDL[R(¹³C6; ¹⁵N4)] | 221 |
| G3IBF4_1* | LPVLLLGR | 106 | LPVLLLG[R(¹³C6; ¹⁵N4)] | 222 |
| G3IBF4_2 | VTAGISFAIPSDK | 107 | VTAGISFAIPSD[K(¹³C6; ¹⁵N2)] | 223 |
| G3IBF4_3* | FLTESHDR | 108 | FLTESHD[R(¹³C6; ¹⁵N4)] | 224 |
| G3IBF4_4 | DFPDVLSGAYIIEVIPDTP | 109 | DFPDVLSGAYIIEVIPDTPAEAGGL[K(¹³C6; | 225 |
| | AEAGGLK | | ¹⁵N2)] | |
| A0A3L7HS03_1* | ELAVAAAYQSVR | 110 | ELAVAAAYQSV[R(¹³C6; ¹⁵N4)] | 226 |
| A0A3L7HS03_2* | WHQTADFGFVPNLK | 111 | WHQTADFGFVPNL[K(¹³C6; ¹⁵N2)] | 227 |
| A0A3L7HS03_3* | LLSLTYDQK | 112 | LLSLTYDQ[K(¹³C6; ¹⁵N2)] | 228 |
| A0A3L7HS03_4* | SFEIS[C(CAM)][C(CAM)] | 113 | SFEIS[C(CAM)][C(CAM)]SD | 229 |
| | SDR | | [R(¹³C6; ¹⁵N4)] | |
| A0A3L7HS03_5 | YAWTTWP[C(CAM)]DYK | 114 | YAWTTWP[C(CAM)]DY[K(¹³C6; ¹⁵N2)] | 230 |
| G3IA12_1* | DNVLHVEINGISPK | 115 | DNVLHVEINGISP[K(¹³C6; ¹⁵N2)] | 231 |
| G3IA12_2* | FSAH[C(CAM)]S[C(CAM)] | 116 | FSAH[C(CAM)]S[C(CAM)]DS[K(¹³C6; | 232 |
| | DSK | | ¹⁵N2)] | |

*Peptides selected for 70 critical HCP AQUA mix

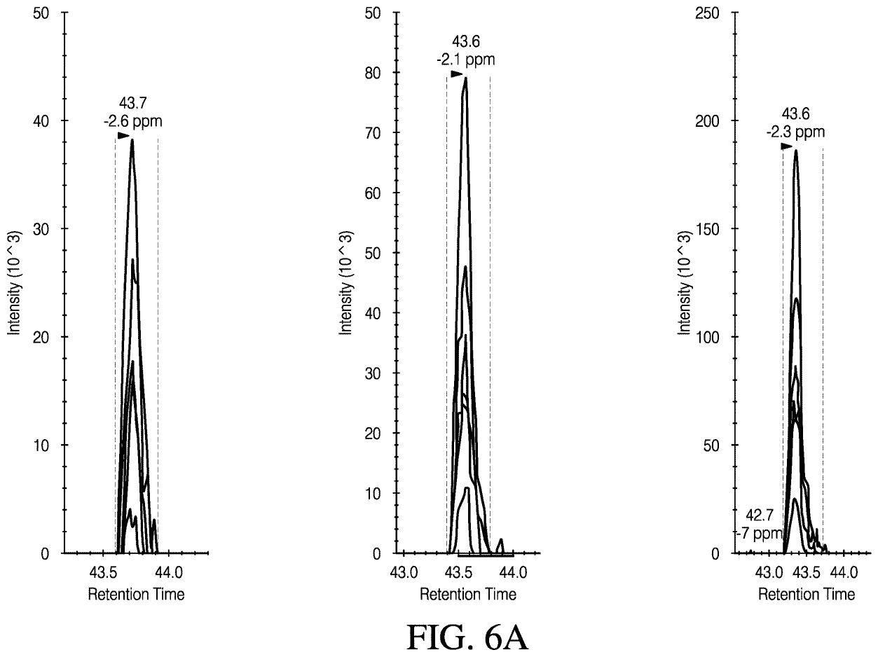

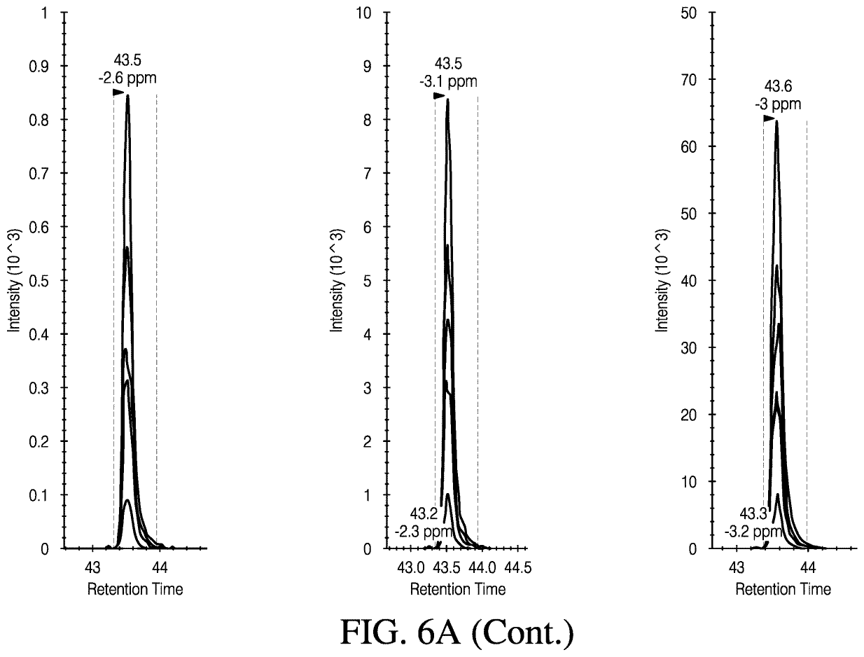

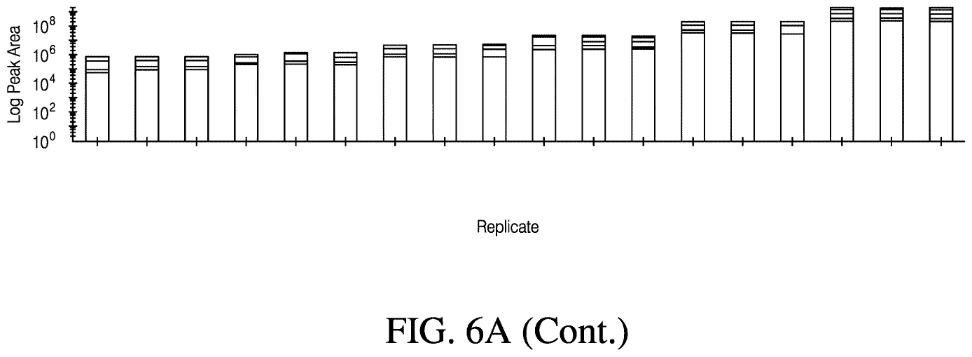

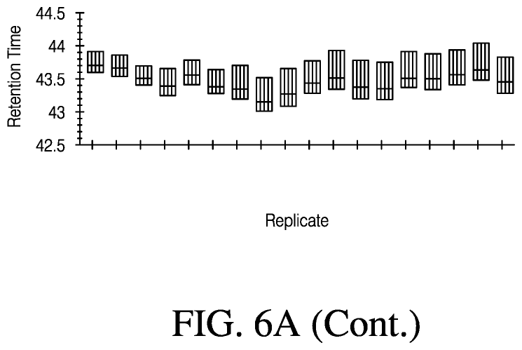

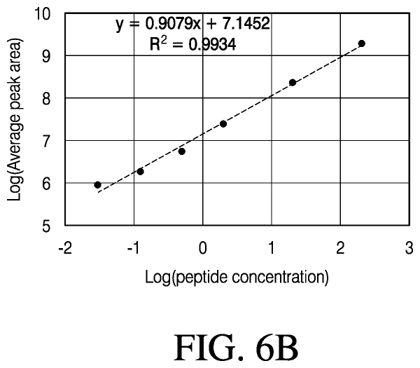

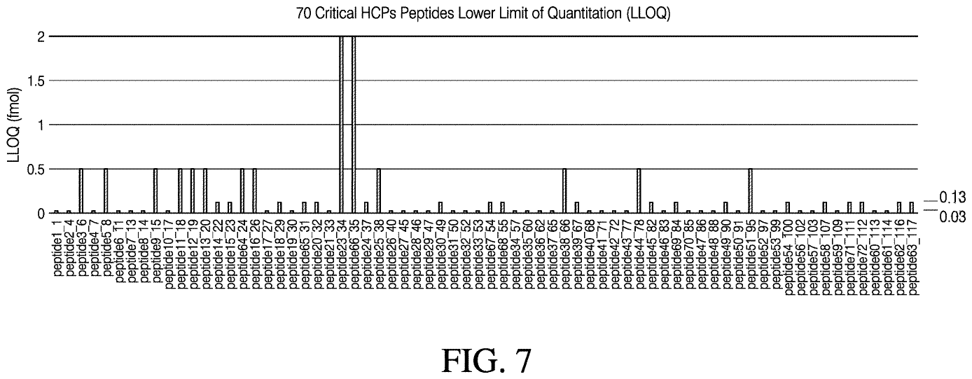

The 70 critical HCPs AQUA peptide mix was used to generate extracted ion chromatograms of fragment ions of each AQUA heavy peptide (FIG. 6A) and six points standard curve spanning from 0.03 to 200 femtomole on column was generated (FIG. 6B). The targeted MS assay allowed linear quantitation and three orders of magnitude dynamic range. Most target peptides were quantified with lower limit of quantitation of 0.03 or 0.13 femtomole (FIG. 7).

### Example 3

### Comparison of Modified Sample Preparation and Native Digestion Methods

### Materials and Methods

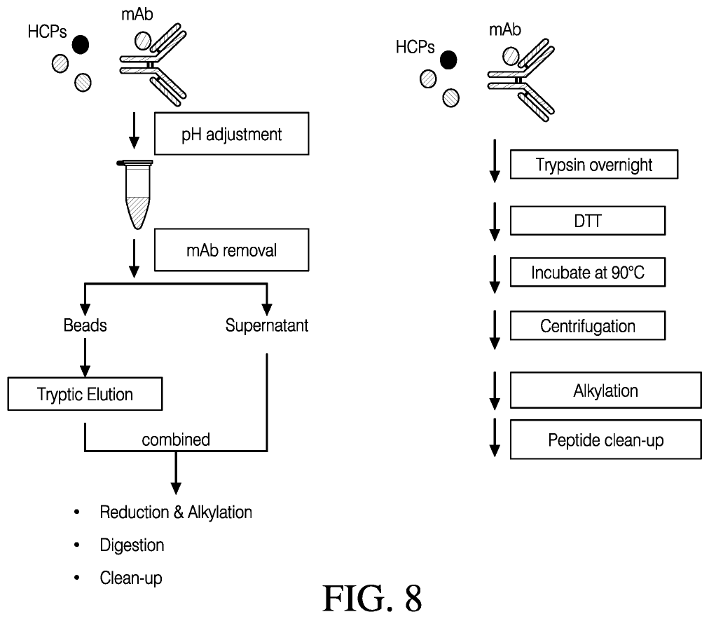

The experiment utilized 1 mg of a one-step affinity purified Herceptin sample as the starting material. To deplete the Herceptin monoclonal antibody, 60 μl of Pierce™ High Capacity Protein A MagBeads, alkali stable (Thermo Fisher Cat. No. A53035) were mixed and incubated with the antibody sample for 1 hour at room temperature with shaking. The magnetic beads were then collected using a magnetic stand, and the supernatant was transferred to a new tube for later use. Trypsin was added to the collected beads for on-beads tryptic elution. Once the tryptic elution was completed, the on-bead tryptic eluates and the previously saved supernatant were combined. The combined tryptic eluate and supernatant were processed using the EasyPep Sample Prep Kit (Thermo Scientific Cat. No. A45734) to effect reduction, alkylation, and clean-up, as illustrated in FIG. 8.

The modified EasyPep sample preparation method was compared to a native digestion sample preparation method. The native digestion method, originally reported by Huang et al. (2017), involved overnight digestion using a low amount of trypsin (1:400 ratio of trypsin-to-protein sample). This resulted in the selective digestion of lower abundance host cell proteins (HCPs), while partially digesting the relatively abundant antibody. To denature the partially digested antibody proteins, a reducing agent (DTT) was added to the overnight digestion sample, followed by heating at 90° C. Subsequent centrifugation effectively removed the partially digested antibody from the sample. The sample was then processed using the EasyPep kit to provide a HCP peptide sample.

The two samples were analyzed using the Quantitative Colorimetric Peptide Assay Kit (Thermo Scientific Cat. No. 23275). This kit allows for the measurement of peptide concentration, which can be used to normalize the samples for subsequent analysis and ensure accurate quantification. For LC-MS/MS analysis, equal amounts of 0.5 μg from each sample were loaded onto the column to ensure that comparable amounts of peptides are analyzed for each sample, allowing for accurate comparison of protein abundance levels. To analyze the abundance levels of the host cell protein (HCP) digest samples, a Thermo Scientific™ Dionex™ UltiMate™ 3000 RSLCnano System coupled to a Thermo Scientific™ Q Exactive™ Plus Mass Spectrometer was used. This LC-MS/MS setup allows for high-resolution and sensitive analysis of the peptides generated from the HCP digest samples, enabling the identification and quantification of proteins present in the samples. Venn diagrams were produced from the MS data using the Proteome Discoverer analysis software package available from Thermo Fisher Scientific (Waltham, MA).

### Results

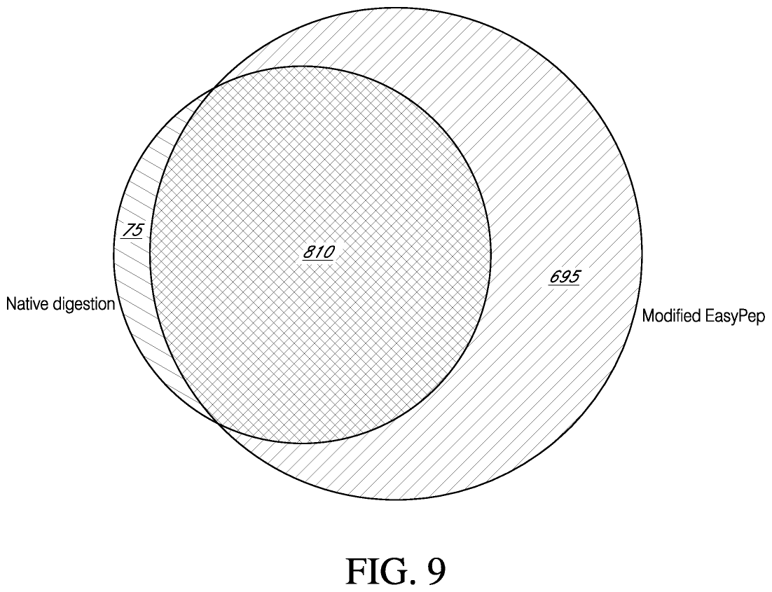

The modified method described above identified a total of 1,505 HCPs in the sample, while the native digestion method identified 885 HCPs, with 810 HCP's identified in both samples (FIG. 9). Table 3 compares the total proteins and unique proteins exclusively identified with either method.

TABLE 3
Comparison of Total Proteins and Unique Host Cell Proteins

| Method | Total Proteins | Unique Proteins |
|---|---|---|
| Native digestion | 885 | 75 |
| Modified EasyPep | 1505 | 695 |

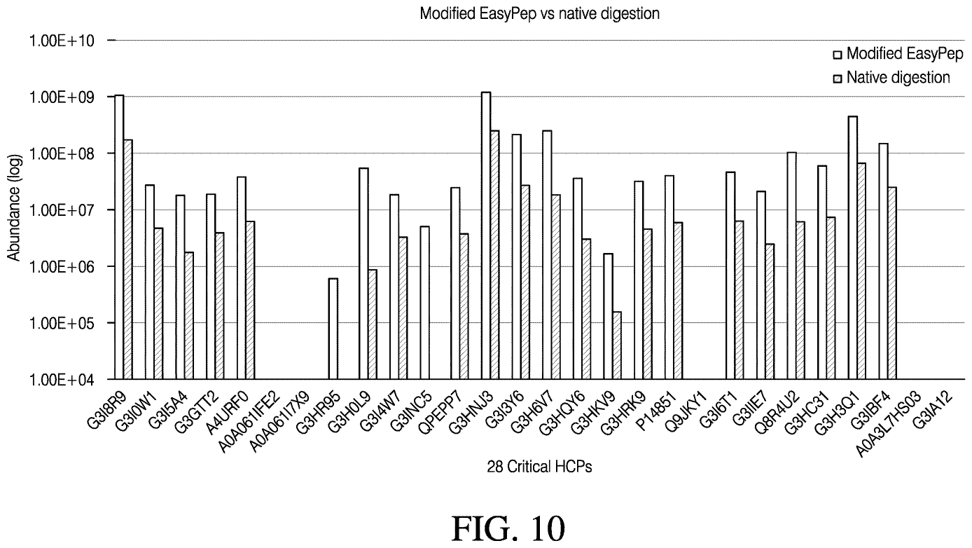

Although both methods identified a similar number of “critical” HCP proteins, with 23 and 21 identified by the modified method and native digestion method, respectively, the abundance levels of these critical HCPs were significantly higher with the modified sample preparation method, where the modified method exhibited one order of magnitude elevation in dynamic range compared to the sample prepared using the native digestion method (FIG. 10).

### Example 4

### Comparison of Modified Sample Preparation and Protein a Depletion and Native Digestion Methods

### Materials and Methods

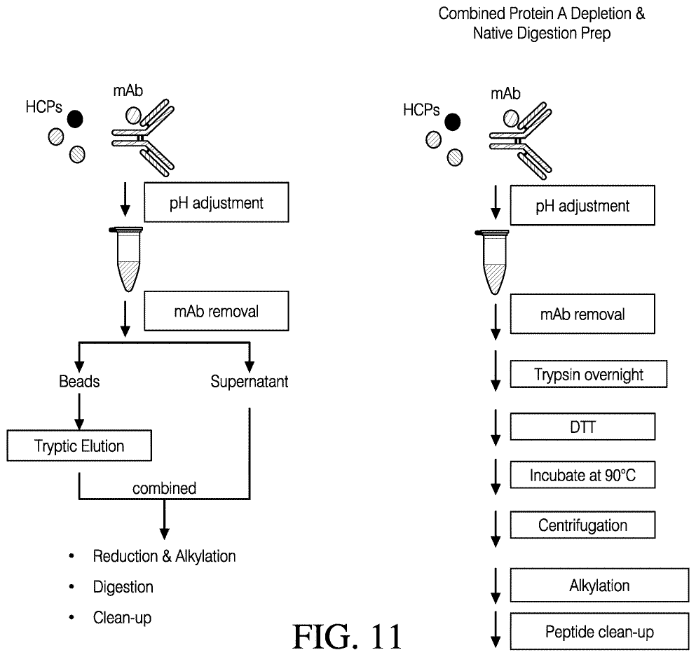

The modified EasyPep sample preparation method was compared to a combination of Protein A depletion and native digestion to evaluate if further improvements in HCP peptide signal could be achieved by reducing interference from the sample (FIG. 11). 1 mg of the one-step affinity purified Herceptin sample was processed and digested using the EasyPep digestion kit, as described in Example 3. In a separate experiment, Protein A MagBeads were used to remove the antibody, as described in Example 3. This was followed by native digestion, which involved overnight digestion with a low amount of trypsin (1:400 ratio of trypsin-to-protein sample). This selective digestion approach aimed to target lower abundance host cell proteins (HCPs), while partially digesting the relatively abundant antibody. To denature the partially digested antibody proteins, a reducing agent (DTT) was added to the overnight digestion sample, followed by heating at 90° C. Subsequent centrifugation effectively removed the partially digested antibody. The sample was then processed using the EasyPep kit to provide an HCP peptide sample. Colorimetric quantification and LC-MS/MS analysis were performed on the three samples as described in Example 3.

### Results

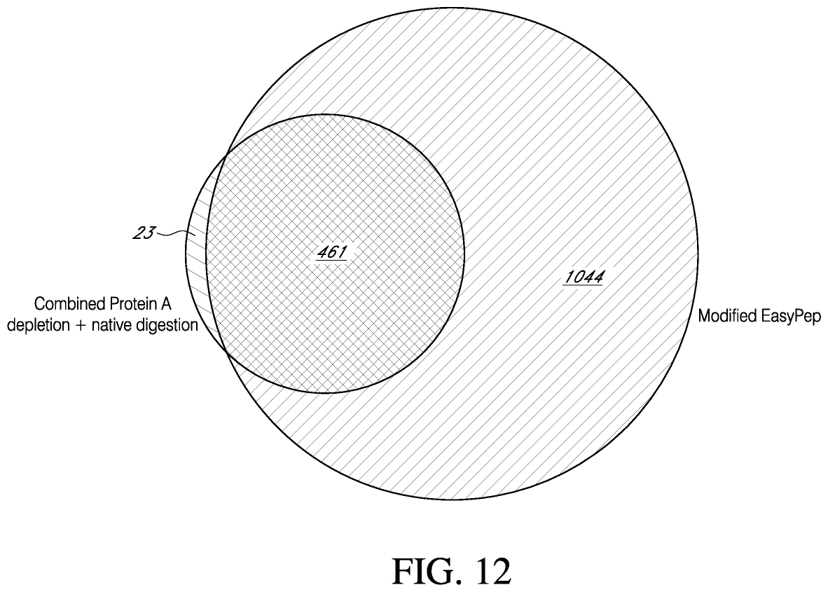

The modified EasyPep sample preparation method successfully identified a total of 1,505 HCPs in the sample. In comparison, the combination of Protein A depletion and native digestion identified 484 HCPs, with 461 HCPs identified in both samples (FIG. 12). Table 4 compares the total proteins and unique proteins exclusively identified with either method.

TABLE 4
Comparison of Total Proteins and Unique Host Cell Proteins

| | Total | |
|---|---|---|
| Method | Proteins | Unique Proteins |
| Protein A depletion + native digestion | 484 | 23 |
| Modified EasyPep | 1505 | 1044 |

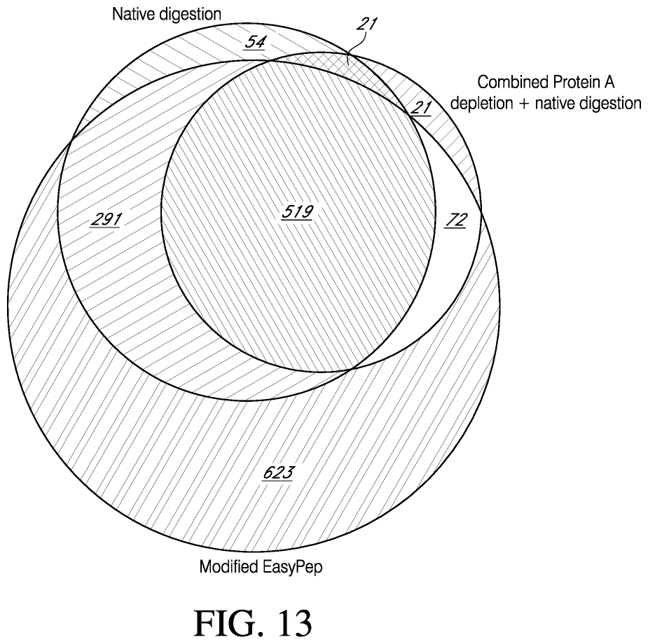

When comparing the three methods (modified EasyPep method, native digestion alone, and native digestion combined with Protein A depletion), it was observed that the modified EasyPep sample preparation method identified more unique proteins than both the native digestion and the combination method (FIG. 13). This suggests that the modified method offers improved performance and sensitivity for the detection and identification of HCPs in the sample. Table 5 compares the total proteins and exclusive proteins for the samples derived from the methods described in this example, with 519 HCPs identified in all three samples.

TABLE 5
Comparison of Total Proteins and Unique Proteins

| | Total | |
|---|---|---|
| Method | Proteins | Unique Proteins |
| Native digestion | 885 | 54 |
| Protein A depletion + native digestion | 633 | 21 |
| Modified EasyPep | 1505 | 623 |

### Example 5

### Detection of Unique HCPs from Supernatant, Beads, and Combination of Supernatant and Beads

### Materials and Methods

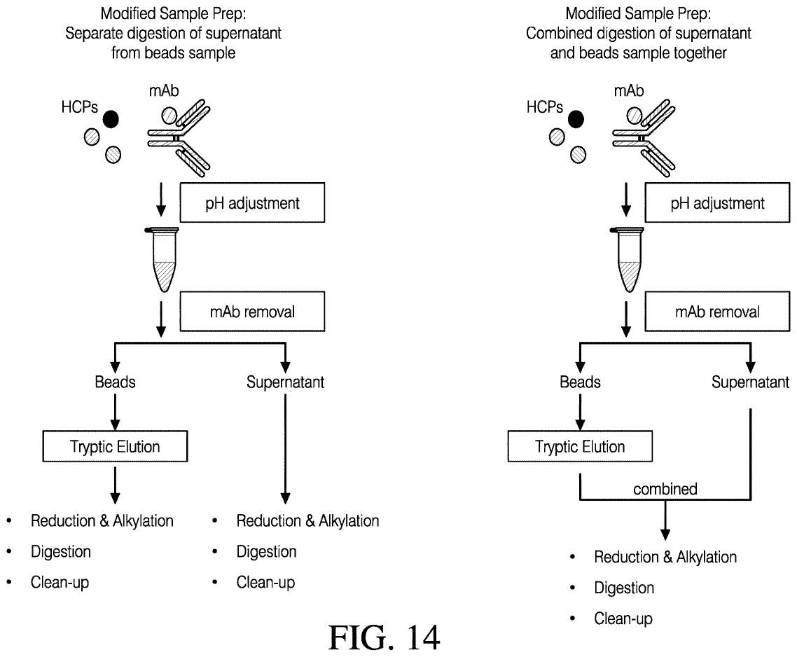

1 mg of the one-step affinity purified Herceptin sample was used as the starting material. To remove the Herceptin monoclonal antibody, 60 μl of Pierce™ High Capacity Protein A MagBeads were incubated with the antibody sample for 1 hour at 23° C. with shaking. The magnetic beads were then collected using a magnetic stand, and the supernatant was transferred to a new tube for digestion at a later stage. For on-beads tryptic elution, trypsin was added to the collected beads. Once the tryptic elution was completed, the EasyPep kit was used to process the supernatant and on-beads tryptic eluate separately, as shown in FIG. 14. This generated two digest samples: supernatant and beads. To compare supernatant and beads samples with the combined sample of supernatant and beads, a separate preparation of the supernatant and beads in combination was made from the one-step affinity purified Herceptin sample as the starting material according to the workflow illustrated in FIG. 14. The three samples (supernatant, beads and combined supernatant and beads) were quantified using the Colorimetric Quantitative Peptide Assay kit and using LC-MS/MS, as described above.

### Results

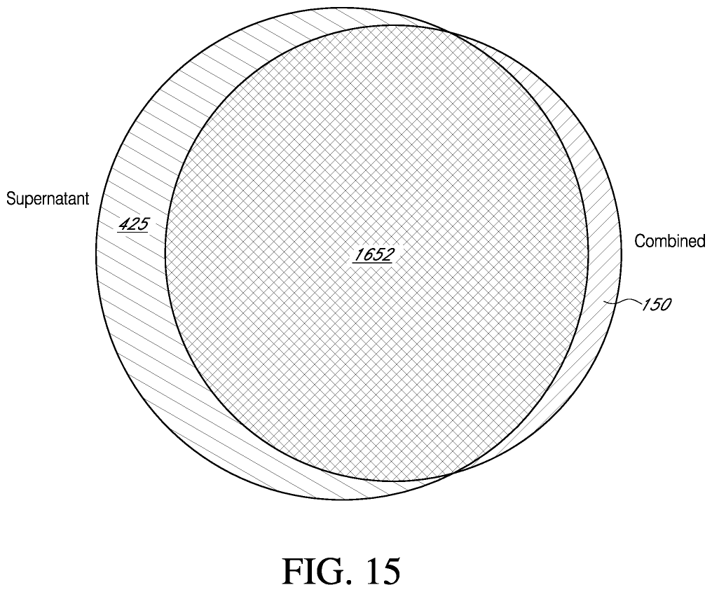

FIG. 15 depicts the results of the MS analysis, indicating that the supernatant identified 10% more HCPs compared to the combined method. The MS analysis identified a total of 2087 host cell proteins (HCPs) from the supernatant sample, while 1812 HCPs were identified from the combined supernatant and beads sample. Among these, 425 unique HCPs were detected in the supernatant sample, whereas 150 unique HCPs were detected in the combined supernatant and beads sample (Table 6). It is noteworthy, however, that a significant number of unique HCPs were detected from the combined sample. This highlights the value of analyzing the combined sample, as it provides valuable information about the proteins that are specifically bound to the Protein A beads during the Protein A depletion step.

TABLE 6
Comparison of Total Proteins and Exclusive Proteins

| | Total | Unique |
|---|---|---|
| Sample Source Method | Proteins | Proteins |
| Supernatant sample from modified sample | 2077 | 425 |
| preparation method | | |
| Combined supernatant and beads from | 1802 | 150 |
| modified sample preparation method | | |
| Supernatant and combined supernatant and | 1652 | 1652 |
| beads sample from modified sample | | |
| preparation method | | |

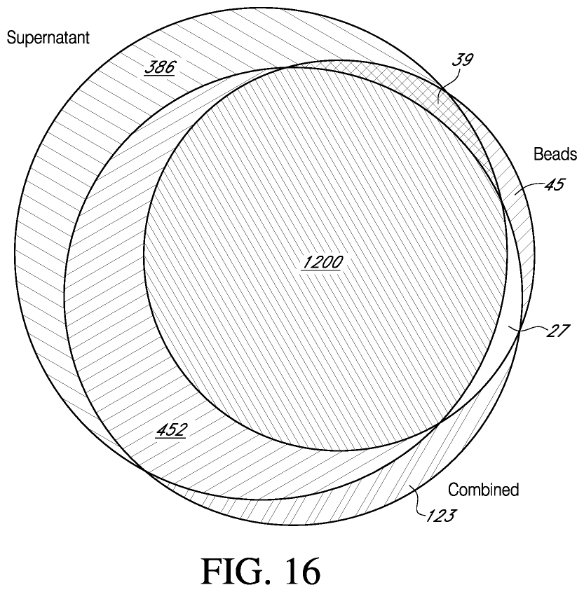

While the supernatant sample showed a higher number of unique HCPs (386), it is still important to identify the significant number of unique HCPs (123) detected in the combined supernatant and beads sample (see, Table 7 and associated FIG. 16). Analyzing the combined sample provides insights into the protein composition and potential contaminants that may be present in the sample without the need to separate the supernatant and beads for individual analysis. This information can help in understanding the overall protein profile and potential impurities present in the sample.

TABLE 7
Comparison of Total Proteins and Exclusive Proteins

| | Total | Unique |
|---|---|---|
| Method | Proteins | Proteins |
| Supernatant separate digestion sample from | 2087 | 386 |
| modified sample preparation method | | |
| Beads separate digestion sample from | 1311 | 45 |
| modified sample preparation method | | |
| Combined digestion of supernatant and | 1802 | 123 |
| beads sample from modified sample | | |
| preparation method | | |
| Supernatant separate digestion, beads | 1200 | 1200 |
| separate digestion, and combined digestion | | |
| of supernatant and beads sample from | | |
| modified sample preparation method | | |

It will be apparent that the precise details of the methods or compositions described may be varied or modified without departing from the spirit of the described aspects of the disclosure. We claim all such modifications and variations that fall within the scope and spirit of the claims below.

1. A method of detecting one or more target proteins in a sample comprising an antibody or fragment thereof, comprising:
contacting the sample with a solid support comprising an immunoglobulin binding protein, wherein the contacting is carried out at a pH of about 8-10, to produce a sample comprising the antibody or antibody fragment bound to the solid support;
contacting the sample comprising the antibody or antibody fragment bound to the solid support with a protease agent to produce partially digested antibody and target proteins;
separating the sample comprising the antibody or antibody fragment bound to the solid support into fractions comprising:
(i) a first fraction comprising the antibody or antibody fragment bound to the solid support, and
(ii) a second fraction comprising a supernatant comprising the partially digested target proteins;
processing the supernatant comprising the partially digested target proteins; and
detecting the one or more target proteins.

2. The method of claim 1, wherein the solid support comprises a bead.

3. (canceled)

4. The method of claim 1, wherein the immunoglobulin binding protein comprises Protein A, Protein G, Protein A/G, or Protein L.

5. (canceled)

6. (canceled)

7. The method of claim 1, wherein processing the supernatant comprising the partially digested target proteins comprises:
reducing and alkylating the partially digested target proteins; and
digesting the reduced and alkylated partially digested target proteins with one or more protease agents to produce digested peptides.

8. The method of claim 7, wherein the one or more protease agents comprises one or more of trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin.

9. (canceled)

10. (canceled)

11. The method of claim 7, wherein detecting the one or more target proteins comprises assaying the digested peptides via mass spectrometry to determine the presence of at least one peptide from the one or more target proteins.

12. (canceled)

13. The method of claim 11, further comprising:
adding one or more internal standard peptides of known amount to the digested peptides prior to mass spectrometry, wherein the one or more internal standard peptides have the same amino acid sequence as a target peptide, and is detectably labeled; and
determining the quantity of the target peptide by comparison to the one or more internal standard peptides.

14. The method of claim 13, wherein the detectable label comprises a mass-altering label.

15. (canceled)

16. The method of claim 7, wherein the target peptide comprises the amino acid sequence of any one of SEQ ID NOs: 1-116.

17. The method of claim 13, wherein the detectably labeled internal standard peptide comprises the amino acid sequence of any one of SEQ ID NOs: 117-232.

18. The method of claim 1, wherein the sample comprising one or more antibodies or antibody fragments is a sample prepared from a host cell culture.

19. The method of claim 1, wherein the one or more target proteins comprises one or more host cell proteins.

20. (canceled)

21. A kit comprising one or more detectably labeled peptides comprising the amino acid sequence of any one of SEQ ID NOs: 1-232.

22. (canceled)

23. The kit of claim 21, wherein the one or more detectably labeled peptides comprise the amino acid sequence of each of SEQ ID NOs: 1, 4, 6, 7, 8, 11, 13, 14, 15, 17, 18, 19, 20, 22, 23, 24, 26, 27, 29, 30, 31, 32, 33, 34, 35, 37, 38, 40, 44, 45, 46, 48, 49, 51, 52, 53, 54, 56, 59, 61, 64, 65, 66, 67, 70, 71, 76, 77, 81, 82, 83, 84, 85, 87, 89, 90, 94, 96, 98, 99, 101, 102, 106, 108, 110, 111, 112, 113, 115, and 116.

24. (canceled)

25. (canceled)

26. The kit of claim 21, wherein the one or more detectably labeled peptides comprise each of SEQ ID NOs: 117, 120, 122, 123, 124, 127, 129, 130, 131, 133, 134, 135, 136, 138, 139, 140, 142, 143, 145, 146, 147, 148, 149, 150, 151, 153, 154, 156, 160, 161, 162, 164, 165, 167, 168, 169, 170, 172, 175, 177, 180, 181, 182, 183, 186, 187, 192, 193, 197, 198, 199, 200, 201, 203, 205, 206, 210, 212, 214, 215, 217, 218, 222, 224, 226, 227, 228, 229, 231, and 232.

27. The kit of claim 21, further comprising a protease agent.

28. The kit of claim 27, wherein the protease agent is trypsin, chymotrypsin, AspN, GluC, LysC, LysN, ArgC, proteinase K, pepsin, clostripain, elastase, GluC biocarb, LysC/P, LysN promisc, protein endopeptidase, Staph protease, or thermolysin.

29. A set of peptides comprising one or more detectably labeled peptides comprising the amino acid sequence of any one of SEQ ID NOs: 1-232.

30. (canceled)

31. The set of peptides of claim 29, wherein the one or more detectably labeled peptides comprise or consist of the amino acid sequence of each of SEQ ID NOs: 1, 4, 6, 7, 8, 11, 13, 14, 15, 17, 18, 19, 20, 22, 23, 24, 26, 27, 29, 30, 31, 32, 33, 34, 35, 37, 38, 40, 44, 45, 46, 48, 49, 51, 52, 53, 54, 56, 59, 61, 64, 65, 66, 67, 70, 71, 76, 77, 81, 82, 83, 84, 85, 87, 89, 90, 94, 96, 98, 99, 101, 102, 106, 108, 110, 111, 112, 113, 115, and 116.

32. (canceled)

33. (canceled)

34. The set of peptides of claim 29, wherein the one or more detectably labeled peptides comprise or consist of each of SEQ ID NOs: 117, 120, 122, 123, 124, 127, 129, 130, 131, 133, 134, 135, 136, 138, 139, 140, 142, 143, 145, 146, 147, 148, 149, 150, 151, 153, 154, 156, 160, 161, 162, 164, 165, 167, 168, 169, 170, 172, 175, 177, 180, 181, 182, 183, 186, 187, 192, 193, 197, 198, 199, 200, 201, 203, 205, 206, 210, 212, 214, 215, 217, 218, 222, 224, 226, 227, 228, 229, 231, and 232.

35. (canceled)
